(19) United States
(12) Patent Application Publication
(10) Pub. No.: US 2008/0279785 A1
(43) Pub. Date: Nov. 13, 2008

(54) Novel synthetic agonists of toll-like receptors containing CG dinucleotide modifications

(76) Inventors: Ekambar R. Kandimalla, Southboro, MA (US); Malikarjuna Reddy Putta, Burlington, MA (US); Yukui Li, Newton, MA (US); Lakshmi Bhagat, Framingham, MA (US); FuGang Zhu, Bedford, MA (US); Sudhir Agrawal, Shrewsbury, MA (US)

Correspondence Address: KEOWN & ZUCCHERO, LLP, 500 WEST CUMMINGS PARK, SUITE 1200, WOBURN, MA, 01801, US

(21) Appl. No.: 11/641,590

(22) Filed: Dec. 19, 2006

Related U.S. Application Data
(60) Provisional application No. 60/752,335, filed on Dec. 20, 2005.
(60) Provisional application No. 60/821,458, filed on Aug. 4, 2006.

(51) Int. Cl. A61K 9/12; C07H 21/02; A61K 39/395; A61K 39/00; C07H 21/04; A61P 35/00; A61K 39/35; A61K 38/02; A61K 31/711

(52) U.S. Cl. 424/45; 536/23.1; 514/44; 424/130.1; 514/2; 424/184.1; 424/275.1; 536/25.6

(57) ABSTRACT

The invention relates to the therapeutic use of oligonucleotides as immune modulatory agents in immunotherapy applications. More particularly, the invention provides immune modulatory oligonucleotide compositions for use in methods for generating an immune response or for treating a patient in need of immune modulation. The immune modulatory oligonucleotides of the invention preferably comprise novel pyrimidines and purines.

### RELATED APPLICATIONS

This application claims the benefit of U.S. Provisional Application Ser. No. 60/752,335, filed on Dec. 20, 2005 and U.S. Provisional Application Ser. No. 60/821,458, filed Aug. 4, 2006. The entire teachings of the above-referenced Applications are incorporated herein by reference.

### BACKGROUND OF THE INVENTION

1. Field of the Invention

The invention generally relates to the field of immunology and immunotherapy applications using oligonucleotides as immune modulatory agents. More specifically, the invention relates to novel chemical compositions and methods of use thereof. Such compositions are effective at generating unique cytokine/chemokine profiles through a TLR9 mediated immune response.

2. Summary of the Related Art

The immune response involves both an innate and an adaptive response based upon the subset of cells involved in the response. For example, the T helper (Th) cells involved in classical cell-mediated functions such as delayed-type hypersensitivity and activation of cytotoxic T lymphocytes (CTLs) are Th1 cells, whereas the Th cells involved as helper cells for B-cell activation are Th2 cells. The type of immune response is influenced by the cytokines produced in response to antigen exposure. Differences in the cytokines secreted by Th1 and Th2 cells may be the result of the different biological functions of these two subsets.

Th1 cells are involved in the body's innate response to antigen (e.g. viral infections, intracellular pathogens, and tumor cells). The result is a secretion of IL-2 and IFN-gamma and a concomitant activation of CTLs. Th2 cells are known to be activated in response to bacteria and parasites and may mediate the body's adaptive immune response (e.g. IgE production and eosinophil activation) through the secretion of IL-4 and IL-5.

The Th1 immune response can be induced in mammals for example by introduction of bacterial or synthetic DNA containing unmethylated CpG dinucleotides, which immune response results from presentation of specific oligonucleotide sequences (e.g. unmethylated CpG) to receptors on certain immune cells known as pattern recognition receptors (PRRs). Certain of these PRRs are Toll-like receptors (TLRs).

Toll-like receptors (TLRs) are intimately involved in the innate immune response. In vertebrates, a family of ten proteins called Toll-like receptors (TLR1 to TLR10) is known to recognize pathogen associated molecular patterns. Of the ten, TLR3, 7, 8, and 9 are known to localize in endosomes inside the cell and recognize nucleic acids (DNA and RNA) and small molecules such as nucleosides and nucleic acid metabolites. TLR3 and TLR9 are known to recognize nucleic acid such as dsRNA and unmethylated CpG dinucleotide present in viral and bacterial and synthetic DNA, respectively. Bacterial DNA has been shown to activate immune system and antitumor activity (Tokunaga T et al., J. Natl. Cancer Inst. (1984) 72:955-962; Shimada S, et al., Jpn. H cancer Res, 1986, 77, 808-816; Yamamoto S, et al., Jpn. J. Cancer Res., 1986, 79, 866-73). Other studies using antisense oligonucleotides containing CpG dinucleotides have been shown to stimulate immune responses (Zhao Q, et al., Biochem. Pharmacol. 1996, 26, 173-82) Subsequent studies showed that TLR9 recognizes unmethylated CpG motifs present in bacterial and synthetic DNA (Hemmi H, Takeuchi O, Kawai T, Kaisho T, Sato S, Sanjo H, Matsumoto M, Hoshino K, Wagner H, Takeda K, Akira S. A Toll-like receptor recognizes bacterial DNA. Nature. (2000); 408:740-5). Other modifications of CpG-containing phosphorothioate oligonucleotides can also affect their ability to act as modulators of immune response through TLR9 (see, e.g., Zhao et al., Biochem. Pharmacol. (1996) 51:173-182; Zhao et al., Biochem Pharmacol. (1996) 52:1537-1544; Zhao et al., Antisense Nucleic Acid Drug Dev. (1997) 7:495-502; Zhao et al., Bioorg. Med. Chem. Lett. (1999) 9:3453-3458; Zhao et al., Bioorg. Med. Chem. Lett. (2000) 10:1051-1054; Yu et al., Bioorg. Med. Chem. Lett. (2000) 10:2585-2588; Yu et al., Bioorg. Med. Chem. Lett. (2001) 11:2263-2267; and Kandimalla et al., Bioorg. Med. Chem. (2001) 9:807-813). In addition, structure activity relationship studies have allowed identification of synthetic motifs and novel DNA-based structures that induce specific immune response profiles that are distinct from those resulting from unmethylated CpG dinucleotides. [Kandimalla E R, Bhagat L, Li Y, Yu D, Wang D, Cong Y P, Song S S, Tang J X, Sullivan T. Agrawal S. Proc Natl Acad Sci USA. 2005; 102:6925-30. Kandimalla E R, Bhagat L, Zhu F G, Yu D, Cong Y P, Wang D, Tang J X, Tang J Y, Knetter C F, Lien E, Agrawal S. Proc Natl Acad Sci USA. 2003; 100:14303-8. Cong Y P, Song S S, Bhagat L, Pandey R K, Yu D, Kandimalla E R, Agrawal S. Biochem Biophys Res Commun. 2003; 310:1133-9. Kandimalla E R, Bhagat L, Cong Y P, Pandey R K, Yu D, Zhao Q, Agrawal S. Biochem Biophys Res Commun. 2003; 306:948-53. Kandimalla E R, Bhagat L, Wang D, Yu D, Zhu F G, Tang J, Wang H, Huang P, Zhang R, Agrawal S, Nucleic Acids Res. 2003; 31:2393-400. Yu D, Kandimalla E R, Zhao Q, Bhagat L, Cong Y, Agrawal S. Bioorg Med. Chem. 2003; 11:459-64. Bhagat L, Zhu F G, Yu D, Tang J, Wang H, Kandimalla E R, Zhang R, Agrawal S. Biochem Biophys Res Commun. 2003; 300:853-61. Yu D, Kandimalla E R, Bhagat L, Tang J Y, Cong Y, Tang J, Agrawal S, Nucleic Acids Res. 2002; 30:4460-9. Yu D, Kandimalla E R, Cong Y, Tang J, Tang J Y, Zhao Q, Agrawal S. J Med Chem. 2002; 45:4540-8. Yu D, Zhu F G, Bhagat L, Wang H, Kandimalla E R, Zhang R, Agrawal S. Biochem Biophys Res Commun. 2002; 297:83-90. Kandimalla E R, Bhagat L, Yu D, Cong Y, Tang J, Agrawal S. Bioconjug Chem. 2002; 13:966-74. Yu D, Kandimalla E R, Zhao Q, Cong Y, Agrawal S, Nucleic Acids Res. 2002; 30:1613-9. Yu D, Kandimalla E R, Zhao Q, Cong Y, Agrawal S. Bioorg Med Chem. 2001; 9:2803-8. Yu D, Kandimalla E R, Zhao Q, Cong Y, Agrawal S. Bioorg Med Chem Lett. 2001; 11:2263-7. Kandimalla E R, Yu D, Zhao Q, Agrawal S. Bioorg Med Chem. 2001; 9:807-13. Yu D, Zhao Q, Kandimalla E R, Agrawal S. Bioorg Med Chem Lett. 2000; 10:2585-8, Putta M R, Zhu F, Li Y, Bhagat L, Cong Y, Kandimalla E R, Agrawal S, Nucleic Acids Res. 2006, 34:3231-8]. In addition, other modifications of CpG-containing phosphorothioate oligonucleotides can also affect their ability to act as modulators of immune response. See, e.g., Zhao et al., Biochem. Pharmacol. (1996) 51:173-182; Zhao et al., Biochem Pharmacol. (1996) 52:1537-1544; Zhao et al., Antisense Nucleic Acid Drug Dev. (1997) 7:495-502; Zhao et al., Bioorg. Med. Chem. Lett. (1999) 9:3453-3458; Zhao et al., Bioorg. Med. Chem. Lett. (2000) 10:1051-1054; Yu et al., Bioorg. Med. Chem. Lett. (2000) 10:2585-2588; Yu et al., Bioorg. Med. Chem. Lett. (2001) 11:2263-2267; and Kandimalla et al., Bioorg. Med. Chem. (2001) 9:807-813.

Oligonucleotides and oligodeoxynucleotides have been used in a wide variety of fields, including but not limited to diagnostic probing, PCR priming, antisense inhibition of gene expression, siRNA, aptamers, ribozymes, and immunotherapeutic agents based on Toll-like Receptors (TLR's). More recently, many publications have demonstrated the use of oligodeoxynucleotides as immune modulatory agents and their use alone or as adjuvants in immunotherapy applications for many diseases, such as allergy, asthma, autoimmunity, cancer, and infectious disease.

These reports make clear that there remains a need to create new chemical entities that are able to generate unique immune responses. However, a challenge remains to generate novel chemical entities that generate unique cytokine/chemokine-mediated immune responses and that are still recognized as ligands for TLR9. Ideally, this challenge might be met through the incorporation of unique chemical bases into the novel chemical entity, which results in new immunotherapic agents and generate unique cytokine/chemokine profiles following administration.

### BRIEF SUMMARY OF THE INVENTION

The invention provides novel chemical entities and their use for generating a unique cytokine/chemokine-mediated immune response. The novel chemical entities are useful for modulating the immune response caused by oligonucleotide compounds. The methods according to the invention enable modifying the cytokine/chemokine profile produced by immune modulatory oligonucleotides for immunotherapy applications. The present inventors have surprisingly discovered that modification of immune modulatory dinucleotides allows flexibility in the profile of the immune response produced.

In a first aspect the invention provides an immune modulatory oligonucleotide comprising an immune stimulatory dinucleotide of the formula CG, wherein C is cytosine, 2′-deoxycytosine, N³-methyl-dC, dF or Ψ-iso-dC, and G is guanosine, 2′-deoxyguanosine or N¹-methyl-dG, provided that when C is cytosine or 2′-deoxycytosine, G is N¹-methyl-dG, and further provided that when G is guanosine or 2′-deoxyguanosine, C is N³-methyl-dC, dF or Ψ-iso-dC.

In a second aspect the invention provides pharmaceutical compositions. These compositions comprise a composition disclosed in the first aspect of the invention and a pharmaceutically acceptable carrier.

In a third aspect the invention provides a method for generating an immune response in a vertebrate, the method comprising administering to the vertebrate an immune modulatory oligonucleotide according to the first or second aspects of the invention.

In a fourth aspect the invention provides a method for therapeutically treating a vertebrate having cancer, an autoimmune disorder, airway inflammation, inflammatory disorders, skin disorders, allergy, asthma or a disease caused by a pathogen, such method comprising administering to the patient an immune modulatory oligonucleotide according to the first or second aspects of the invention.

In a fifth aspect the invention provides a method for preventing cancer, an autoimmune disorder, airway inflammation, inflammatory disorders, skin disorders, allergy, asthma or a disease caused by a pathogen in a vertebrate, such method comprising administering to the vertebrate an immune modulatory oligonucleotide according to the first or second aspects of the invention.

### BRIEF DESCRIPTION OF THE DRAWINGS

FIG. 1 depicts a group of representative small molecule linkers suitable for linear synthesis of immune modulatory oligonucleotides of the invention.

FIG. 2 depicts a group of representative small molecule linkers suitable for parallel synthesis of immune modulatory oligonucleotides of the invention.

FIG. 3 is a synthetic scheme for the linear synthesis of immune modulatory oligonucleotides of the invention. DMTr=4,4′-dimethoxytrityl; CE=cyanoethyl.

FIG. 4 is a synthetic scheme for the parallel synthesis of immune modulatory oligonucleotides of the invention. DMTr=4,4′-dimethoxytrityl; CE=cyanoethyl.

FIGS. 5A-5D show IL-12 and IL-6 levels in C57BL/6 mouse spleen cell cultures after administration of immune modulatory oligonucleotides according to the invention. FIGS. 5A-5D more generally demonstrates that the administration of immune modulatory oligonucleotides containing novel bases generates unique IL-12 and IL-6 profiles.

FIGS. 6A and 6B show IL-6 and IL-10 levels in human PBMC cultures after administration of immune modulatory oligonucleotides according to the invention. FIGS. 6A-6B more generally demonstrate that administration of immune modulatory oligonucleotides containing novel bases generates unique IL-6 and IL-10 profiles.

FIG. 7 shows TLR9 activation in HEK293 cells, as measured by their NF-kB activity, after administration of immune modulatory oligonucleotides according to the invention. FIG. 7 more generally demonstrates that administration of immune modulatory oligonucleotides containing novel bases generates unique TLR9 activation profiles.

FIG. 8 shows IL-12 levels in C57BL/6 mice after subcutaneous (s.c.) administration of immune modulatory oligonucleotides according to the invention. FIG. 8 more generally demonstrates that administration in vivo of immune modulatory oligonucleotides containing novel bases generates unique IL-12 profiles.

FIG. 9 shows the spleen weight in C57BL/6 mice after administration of immune modulatory oligonucleotides according to the invention. FIG. 9 more generally demonstrates that administration in vivo of immune modulatory oligonucleotides containing novel bases generates unique immune response profiles.

FIGS. 10A-10D show IL-5, IL-12, IL-13 and IFN-γ levels in OVA-sensitized mouse spleen cells after administration of immune modulatory oligonucleotides according to the invention. FIGS. 10A-10D more generally demonstrate that administration of immune modulatory oligonucleotides containing novel bases generates unique cytokine/chemokine profiles, even in the presence of an immune system activator (e.g. ovalbumin), which vary with the base composition and the amount of the oligonucleotide administered.

FIG. 11 demonstrates activation of HEK293 cells expressing mouse TLR9 with immune modulatory oligonucleotides and control compounds at a concentration of 10 μg/ml. FIG. 11 more generally demonstrates that administration of immune modulatory oligonucleotides containing novel bases generates unique TLR9 activation profiles.

FIGS. 12A-12B demonstrate induction of cytokine secretion by immune modulatory oligonucleotides according to the invention in C57BL/6 mouse spleen cell cultures. FIGS. 12A-12B more generally demonstrate that administration of immune modulatory oligonucleotides containing novel bases generates unique IL-6 and IL-12 profiles, which vary with the base composition and the amount of the oligonucleotide administered.

FIGS. 13A and 13B demonstrate Splenomegaly (FIG. 13A) 72 h after animals received immune modulatory oligonucleotide, control compound, or PBS administered s.c., and (FIG. 13B) IL-12 secretion induced by immune modulatory oligonucleotides following s.c. administration. FIGS. 13A-13B more generally demonstrate that administration in vivo of immune modulatory oligonucleotides containing novel bases generates unique immune response profiles.

FIG. 14 demonstrates Human B-cell proliferation induced by immune modulatory oligonucleotides. FIG. 14 more generally demonstrates that administration of immune modulatory oligonucleotides containing novel bases generates unique cell proliferation profiles, which vary with the base composition and the amount of the oligonucleotide administered.

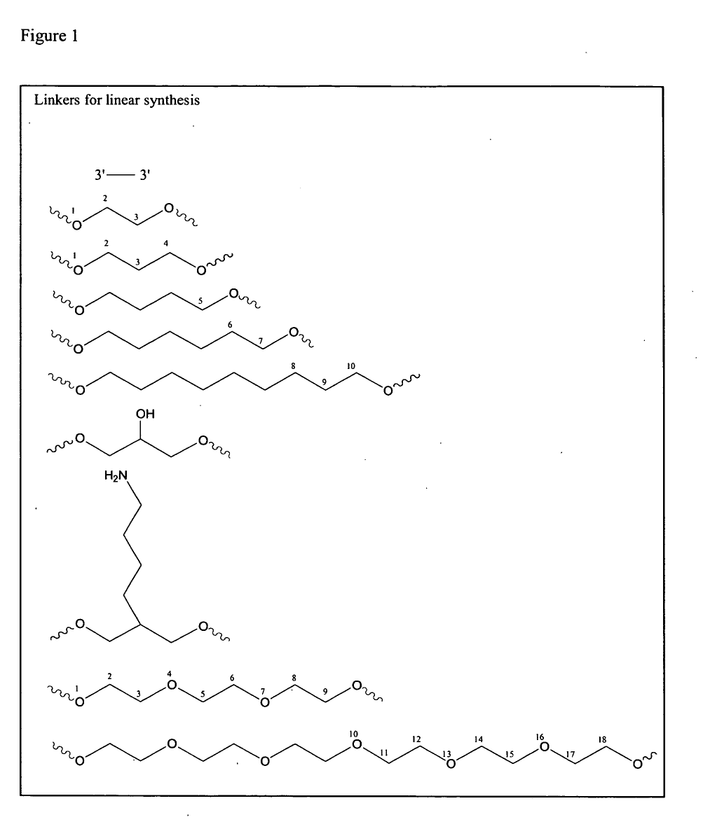

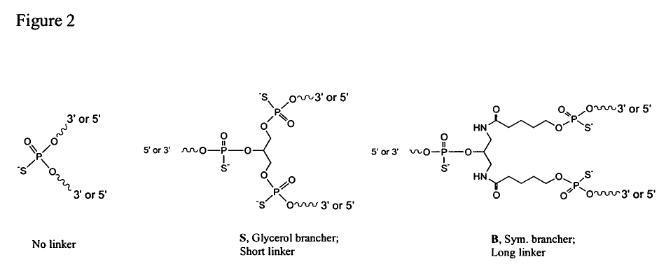

### DETAILED DESCRIPTION OF VARIOUS EMBODIMENTS

The invention relates to the therapeutic use of oligonucleotides as immune modulatory agents for immunotherapy applications. The issued patents, patent applications, and references that are cited herein are hereby incorporated by reference to the same extent as if each was specifically and individually indicated to be incorporated by reference. In the event of inconsistencies between any teaching of any reference cited herein and the present specification, the latter shall prevail for purposes of the invention.

The invention provides methods for enhancing the immune response caused by immune stimulatory compounds used for immunotherapy applications such as, but not limited to, treatment of cancer, autoimmune disorders, asthma, respiratory allergies, food allergies, and bacteria, parasitic, and viral infections in adult and pediatric human and veterinary applications. Thus, the invention further provides compounds having optimal levels of immune stimulatory effect for immunotherapy and methods for making and using such compounds. In addition, compounds of the invention are useful as adjuvants in combination with DNA vaccines, antibodies, and allergens; and in combination with chemotherapeutic agents and/or antisense oligonucleotides.

In a first aspect, the invention provides an immune modulatory oligonucleotide comprising at least one immune modulatory dinucleotide of the formula CG, wherein C is cytosine, 2′-deoxycytosine, N³-methyl-dC, dF or Ψ-iso-dC, and G is guanosine, 2′-deoxyguanosine, 2′-deoxy-7-deazaguanosine, arabinoguanosine or N¹-methyl-dG, provided that when C is cytosine or 2′-deoxycytosine, G is N′-methyl-dG, and further provided that when G is guanosine or 2′-deoxyguanosine, C is N³-methyl-dC, dF or Ψ-iso-dC.

In one embodiment of this aspect, the invention provides immune modulatory oligonucleotides alone or comprising at least two oligonucleotides linked at their 3′ ends, or an internucleoside linkage or a functionalized nucleobase or sugar to a non-nucleotidic linker, at least one of the oligonucleotides being an immune modulatory oligonucleotide and having an accessible 5′ end. The oligonucleotides linked to each other through a non-nucleotidic linker can have an identical nucleotide sequence or can have different nucleotide sequences, provided that at least one of the oligonucleotides contains at least one immune modulatory dinucleotide of the invention.

As used herein, the term “accessible 5′ end” means that the 5′ end of the oligonucleotide is sufficiently available such that the factors that recognize and bind to oligonucleotide and stimulate the immune system have access to it. In oligonucleotides having an accessible 5′ end, the 5′ OH position of the terminal sugar is not covalently linked to more than two nucleoside residues or any other moiety that interferes with interaction with the 5′ end. Optionally, the 5′ OH can be linked to a phosphate, phosphorothioate, or phosphorodithioate moiety, an aromatic or aliphatic linker, cholesterol, or another entity which does not interfere with accessibility.

For purposes of the invention, the term “immune stimulatory oligonucleotide” or “immune modulatory oligonucleotide” means a compound comprising at least one immune modulatory dinucleotide, without which the compound would not have an immune modulatory effect. An “immune modulatory dinucleotide” is a dinucleotide having the formula 5′-CpG-3′, wherein “C” is a pyrimidine nucleoside naturally occurring in mammals or a synthetic derivative thereof and “G” is a purine nucleoside naturally occurring in mammals or a synthetic derivative thereof. The immune modulatory oligonucleotides according to the invention can have one immune modulatory dinucleotide or several immune modulatory dinucleotides. For example, each immune modulatory oligonucleotide can have 2, 3, 4 or more immune modulatory dinucleotides which are identical or can independently be modified as described herein.

The terms “CpG” and “CpG dinucleotide” refer to the dinucleotide 5′-deoxycytidine-deoxyguanosine-3′, wherein p is an internucleoside linkage including, but not limited to, phosphodiester, phosphorothioate and phosphorodithioate linkages.

For purposes of the invention, the term “oligonucleotide” refers to a polynucleoside formed from a plurality of linked nucleoside units. Such oligonucleotides can be obtained from existing nucleic acid sources, including genomic or cDNA, but are preferably produced by synthetic methods. In some embodiments each nucleoside unit includes a heterocyclic base and a pentofuranosyl, trehalose, arabinose, 2′-deoxy-2′-substituted arabinose, 2′-O-substituted arabinose or hexose sugar group. The nucleoside residues can be coupled to each other by any of the numerous known internucleoside linkages. Such internucleoside linkages include, without limitation, phosphodiester, phosphorothioate, phosphorodithioate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. The term “oligonucleotide” also encompasses polynucleosides having one or more stereospecific internucleoside linkage (e.g., (R_{P})- or (S_{P})-phosphorothioate, alkylphosphonate, or phosphotriester linkages). As used herein, the terms “oligonucleotide” and “dinucleotide” are expressly intended to include polynucleosides and dinucleosides having any such internucleoside linkage, whether or not the linkage comprises a phosphate group. In certain embodiments, these internucleoside linkages may be phosphodiester, phosphorothioate, or phosphorodithioate linkages, or combinations thereof.

In some embodiments, the oligonucleotides each have from about 3 to about 35 nucleoside residues, or from about 4 to about 30 nucleoside residues, or from about 4 to about 18 nucleoside residues. In some embodiments, the immune modulatory oligonucleotides comprise oligonucleotides have from about 1 to about 18, or from about 1 to about 15, or from about 5 to about 14, nucleoside residues. As used herein, the term “about” implies that the exact number is not critical. Thus, the number of nucleoside residues in the oligonucleotides is not critical, and oligonucleotides having one or two fewer nucleoside residues, or from one to several additional nucleoside residues are contemplated as equivalents of each of the embodiments described above. In some embodiments, one or more of the oligonucleotides have 11 nucleotides or 18 nucleotides. In the context of immune modulatory oligonucleotides, certain embodiments have from about 13 to about 35 nucleotides, or from about 13 to about 26 nucleotides, or from about 11 to about 22 nucleotides.

The term “oligonucleotide” also encompasses polynucleosides having additional substituents including, without limitation, protein groups, lipophilic groups, intercalating agents, diamines, folic acid, cholesterol and adamantane. The term “oligonucleotide” also encompasses any other nucleobase containing polymer, including, without limitation, peptide nucleic acids (PNA), peptide nucleic acids with phosphate groups (PHONA), morpholino-backbone oligonucleotides, and oligonucleotides having backbone sections with alkyl linkers or amino linkers.

The oligonucleotides of the invention can include naturally occurring nucleosides, modified nucleosides, or mixtures thereof. As used herein, the term “modified nucleoside” is a nucleoside that includes a modified heterocyclic base, a modified sugar moiety, or a combination thereof. In some embodiments, the modified nucleoside is a non-natural pyrimidine or purine nucleoside, as herein described. In some embodiments, the modified nucleoside is a 2′-substituted ribonucleoside, an arabinonucleoside or a 2′-deoxy-2′-substituted-arabinoside.

For purposes of the invention, the term “2′-substituted ribonucleoside” or “2′-substituted arabinoside” includes ribonucleosides or arabinonucleoside in which the hydroxyl group at the 2′ position of the pentose moiety is substituted to produce a 2′-substituted or 2′-O-substituted ribonucleoside. Such substitution is with a lower alkyl group containing 1-6 saturated or unsaturated carbon atoms, or with an aryl group having 6-10 carbon atoms, wherein such alkyl, or aryl group may be unsubstituted or may be substituted, e.g., with halo, hydroxy, trifluoromethyl, cyano, nitro, acyl, acyloxy, alkoxy, carboxyl, carboalkoxy, or amino groups. Examples of 2′-O-substituted ribonucleosides or 2′-O-substituted-arabinosides include, without limitation 2′-O-methylribonucleosides or 2′-O-methylarabinosides and 2′-O-methoxyethylribonucleosides or 2′-O-methoxyethylarabinosides.

The term “2′-substituted ribonucleoside” or “2′-substituted arabinoside” also includes ribonucleosides or arabinonucleosides in which the 2′-hydroxyl group is replaced with a lower alkyl group containing 1-6 saturated or unsaturated carbon atoms, or with an amino or halo group. Examples of such 2′-substituted ribonucleosides or 2′-substituted arabinosides include, without limitation, 2′-amino, 2′-fluoro, 2′-allyl, and 2′-propargyl ribonucleosides or arabinosides.

The term “oligonucleotide” includes hybrid and chimeric oligonucleotides. A “chimeric oligonucleotide” is an oligonucleotide having more than one type of internucleoside linkage. One example of such a chimeric oligonucleotide is a chimeric oligonucleotide comprising a phosphorothioate, phosphodiester or phosphorodithioate region and non-ionic linkages such as alkylphosphonate or alkylphosphonothioate linkages (see e.g., Pederson et al. U.S. Pat. Nos. 5,635,377 and 5,366,878).

A “hybrid oligonucleotide” is an oligonucleotide having more than one type of nucleoside. One example of such a hybrid oligonucleotide comprises a ribonucleotide or 2′-substituted ribonucleotide region, and a deoxyribonucleotide region (see, e.g., Metelev and Agrawal, U.S. Pat. Nos. 5,652,355, 6,346,614 and 6,143,881).

For purposes of the invention, the term “immune stimulatory oligonucleotide” or “immune modulatory oligonucleotide” refers to an oligonucleotide as described above that modulates (e.g. induces) an immune response when administered to a vertebrate, such as a fish, fowl, or mammal. As used herein, the term “mammal” includes, without limitation rats, mice, cats, dogs, horses, cattle, cows, pigs, rabbits, non-human primates, and humans.

For purposes of the invention, a “natural” nucleoside is one that includes one of the five commonly occurring bases in DNA or RNA (e.g., adenosine, guanosine, thymidine, cytosine and uridine) with a deoxyribose or ribose sugar. For purposes of the invention, a “modified” or “non-natural” nucleoside is one that includes a modified naturally occurring base and/or a modified naturally occurring sugar moiety. Examples of modified naturally occurring bases include but are not limited to those compositions represented by Formula I or Formula II. For purposes of the invention, a “dinucleotide analog” is an immune stimulatory dinucleotide as described above, wherein either or both of the pyrimidine and purine nucleosides is a non-natural nucleoside. The terms “C*pG” and “CpG*” refer to immune stimulatory dinucleotide analogs comprising a cytidine analog (non-natural pyrimidine nucleoside) or a guanosine analog (non-natural purine nucleoside), respectively.

In various places the dinucleotide is expressed as R′pG, C*pG or YZ, in which case respectively, R′, C*, or Y represents a synthetic or non-natural pyrimidine, such as, but not limited to, N³-methyl-dC, pseudo-iso-deoxycytodine (i.e., ψ-iso-dC) and deoxyfuranosyl (i.e., dF). In other places the dinucleotide is expressed as CpR, CpG* or YZ, in which case respectively, R, G*, or Z represents a synthetic purine, such as, but not limited to, N¹-methyl-dG or 7-deaza-dG. As used herein, the term “pyrimidine nucleoside” refers to a nucleoside wherein the base component of the nucleoside is a monocyclic nucleobase. Similarly, the term “purine nucleoside” refers to a nucleoside wherein the base component of the nucleoside is a bicyclic nucleobase. For purposes of the invention, a “synthetic” pyrimidine or purine nucleoside includes a non-naturally occurring pyrimidine or purine base, a non-naturally occurring sugar moiety, or a combination thereof.

Pyrimidine nucleosides according to the invention have the structure (1):

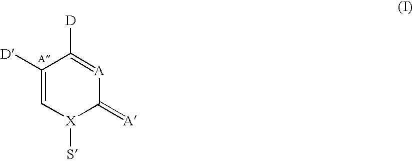

wherein:

D is a hydrogen bond donor;

D′ is selected from the group consisting of hydrogen, hydrogen bond donor, hydrogen bond acceptor, hydrophilic group, hydrophobic group, electron withdrawing group and electron donating group;

D and D′ may be part of a 5-member or 6-member ring;

A is a nitrogen or heteroatom, substituted or unsubstituted heteroatom;

A′ is selected from the group consisting of hydrogen bond acceptor, hydrophilic group, hydrophobic group, electron withdrawing group and electron donating group;

A″ is carbon or nitrogen

X is carbon or nitrogen; and S′ is a pentose or hexose sugar ring, or a non-naturally occurring sugar.

In some embodiments, the sugar ring is derivatized with a phosphate moiety, modified phosphate moiety, or other linker moiety suitable for linking the pyrimidine nucleoside to another nucleoside or nucleoside analog.

Hydrogen bond donors include, without limitation, —NH—, —NH₂, —SH and —OH. Hydrogen bond acceptors include, without limitation, C═O, C═S, and the ring nitrogen atoms of an aromatic heterocycle, e.g., N3 of cytosine.

In some embodiments, the base moiety in (1) is a non-naturally occurring pyrimidine base. Examples of non-naturally occurring pyrimidine bases include, without limitation, 5-hydroxycytosine, 5-hydroxymethylcytosine, N³-methyl-dC, pseudo-iso-deoxycytodine (i.e., ψ-iso-dC); deoxyfuranosyl (i.e., dF), 4-thiouracil and N4-alkylcytosine, such as N4-ethylcytosine. However, in some embodiments 5-bromocytosine is specifically excluded.

In some embodiments, the sugar moiety S′ in (1) is a modified naturally occurring sugar moiety. For purposes of the present invention, a “naturally occurring sugar moiety” is a sugar moiety that occurs naturally as part of nucleic acid, e.g., ribose and 2′-deoxyribose, and a “modified naturally occurring sugar moiety” is any sugar that does not occur naturally as part of a nucleic acid, but which can be used in the backbone for an oligonucleotide, e.g, hexose. Arabinose and arabinose derivatives are examples of sugar moieties.

Purine nucleoside analogs according to the invention have the structure (II):

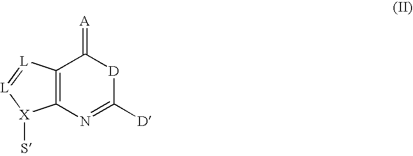

wherein:

D is a nitrogen or heteroatom, substituted or unsubstituted heteroatom;

D′ is selected from the group consisting of hydrogen, hydrogen bond donor, and hydrophilic group;

A is a hydrogen bond acceptor or a hydrophilic group;

X is carbon or nitrogen;

each L is independently an atom selected from the group consisting of C, O, N and S; and

S′ is a pentose or hexose sugar ring, or a non-naturally occurring sugar.

In some embodiments, the sugar ring is derivatized with a phosphate moiety, modified phosphate moiety, or other linker moiety suitable for linking the pyrimidine nucleoside to another nucleoside or nucleoside analog.

Hydrogen bond donors include, without limitation, —NH—, —NH₂, —SH and —OH. Hydrogen bond acceptors include, without limitation, C═O, C═S, —NO₂ and the ring nitrogen atoms of an aromatic heterocycle, e.g., N1 of guanine.

In some embodiments, the base moiety in (II) is a non-naturally occurring purine base. Examples of non-naturally occurring purine bases include, without limitation, 2-amino-6-thiopurine, 7-deazaguanosine, N¹-methyl-dG and 2-amino-6-oxo-7-deazapurine. In some embodiments, the sugar moiety S′ in (II) is a naturally occurring sugar moiety or modified natural occurring sugar moiety, as described above for structure (1).

In some embodiments, the immune stimulatory dinucleotide is selected from the group consisting of C*pG, CpG*, and C*pG*, wherein the base of C is cytosine, the base of C* is thymine, 5-hydroxycytosine, N³-methyl-dC, N4-alkyl-cytosine, pseudo-iso-deoxycytodine; deoxyfuranosyl, 4-thiouracil or other non-natural pyrimidine, or 2-oxo-7-deaza-8-methylpurine, wherein when the base is 2-oxo-7-deaza-8-methyl-purine, it is preferably covalently bound to the 1′-position of a pentose via the 1 position of the base; the base of G is guanosine, the base of G* is 2-amino-6-oxo-7-deazapurine, 2-oxo-7-deaza-8-methylpurine, 6-thioguanine, 7-deazaguanosine, inosine, N₁-methyl-dG, 6-oxopurine, or other non-natural purine nucleoside, and p is an internucleoside linkage selected from the group consisting of phosphodiester, phosphorothioate, and phosphorodithioate, provided that at least one C or G is not cytosine or guanosine, respectively.

The immune modulatory oligonucleotides may include immune stimulatory moieties on one or both sides of the immune stimulatory dinucleotide. Thus, in some embodiments, the immune stimulatory oligonucleotide comprises an immune stimulatory domain of structure (III):

5′-Nn-N1-Y-Z-N1-Nn-3′ (III)

wherein:
the base of Y is cytosine, thymine, 5-hydroxycytosine, N4-alkyl-cytosine, N³-methyl-cytosine, 1-iso-dC, dF, 4-thiouracil or other non-natural pyrimidine nucleoside, or 2-oxo-7-deaza-8 methyl purine, wherein when the base is 2-oxo-7-deaza-8-methyl-purine, it is preferably covalently bound to the 1′-position of a pentose via the 1 position of the base;
the base of Z is guanine, 2-amino-6-oxo-7-deazapurine, 2-oxo-7deaza-8-methylpurine, 2-amino-6-thio-purine, 7-deazaguanosine, N¹-methyl-dG, 6-oxopurine or other non-natural purine nucleoside;

N1 and Nn, independent at each occurrence, is preferably a naturally occurring or a non-natural or synthetic nucleoside or an immune stimulatory moiety selected from the group consisting of abasic nucleosides, N³-methyl-dC, N¹-methyl-dG, arabinonucleosides, 2′-deoxyuridine, α-deoxyribonucleosides, β-L-deoxyribonucleosides, and nucleosides linked by a phosphodiester or modified internucleoside linkage to the adjacent nucleoside on the 3′ side, the modified internucleotide linkage being selected from, without limitation, a linker having a length of from about 2 angstroms to about 200 angstroms, C2-C18 alkyl linker, poly(ethylene glycol) linker, 2-aminobutyl-1,3-propanediol linker, glyceryl linker, 2′-5′ internucleoside linkage, and phosphorothioate, phosphorodithioate, or methylphosphonate internucleoside linkage;

provided that at least one N1 or Nn is optionally an immune stimulatory moiety;

further provided that at least one Y or Z is not cytosine or guanosine, respectively;

wherein n is a number from 0 to 30; and

wherein the 3′ end, an internucleoside linker, or a derivatized nucleobase or sugar is linked directly or via a non-nucleotidic linker to another oligonucleotide, which may or may not be immune stimulatory.

In some embodiments, YZ is cytosine, ψ-iso-dC, dF or N³-methyl-dC and guanosine or N¹-methyl-dG. Immune stimulatory moieties include natural phosphodiester backbones and modifications in the phosphate backbones, including, without limitation, methylphosphonates, methylphosphonothioates, phosphotriesters, phosphothiotriesters, phosphorothioates, phosphorodithioates, triester prodrugs, sulfones, sulfonamides, sulfamates, formacetal, N-methylhydroxylamine, carbonate, carbamate, morpholino, boranophosphonate, phosphoramidates, especially primary amino-phosphoramidates, N3 phosphoramidates and N5 phosphoramidates, and stereospecific linkages (e.g., (R_{P})- or (S_{P})-phosphorothioate, alkylphosphonate, or phosphotriester linkages).

In some embodiments, immune stimulatory oligonucleotides according to the invention further include nucleosides having sugar modifications, including, without limitation, 2′-substituted pentose sugars including, without limitation, 2′-O-methylribose, 2′-O-methoxyethylribose, 2′-O-propargylribose, and 2′-deoxy-2′-fluororibose; 3′-substituted pentose sugars, including, without limitation, 3′-O-methylribose; 1′,2′-dideoxyribose; arabinose; substituted arabinose sugars, including, without limitation, 1′-methylarabinose, 3′-hydroxymethylarabinose, 4′-hydroxymethylarabinose, 3′-hydroxyarabinose and 2′-substituted arabinose sugars; hexose sugars, including, without limitation, 1,5-anhydrohexitol; and alpha-anomers. In embodiments in which the modified sugar is a 3′-deoxyribonucleoside or a 3′-O-substituted ribonucleoside, the immune stimulatory moiety is attached to the adjacent nucleoside by way of a 2′-5′ internucleoside linkage.

In some embodiments, immune stimulatory oligoncucleotides according to the invention further include oligonucleotides having other carbohydrate backbone modifications and replacements, including peptide nucleic acids (PNA), morpholino backbone oligonucleotides, and oligonucleotides having backbone linker sections having a length of from about 2 angstroms to about 200 angstroms, including without limitation, alkyl linkers or amino linkers. The alkyl linker may be branched or unbranched, substituted or unsubstituted, and chirally pure or a racemic mixture. In some embodiments, such alkyl linkers have from about 2 to about 18 carbon atoms. In some embodiments such alkyl linkers have from about 3 to about 9 carbon atoms. Some alkyl linkers include one or more functional groups selected from the group consisting of hydroxy, amino, thiol, thioether, ether, amide, thioamide, ester, urea, and thioether. Some such functionalized alkyl linkers are poly(ethylene glycol) linkers of formula —O—(CH₂—CH₂—O—)ₙ (n=1-9) or glycerol. Some other functionalized alkyl linkers are peptides or amino acids.

In some embodiments, immune stimulatory oligoncucleotides according to the invention further include DNA isoforms, including, without limitation, β-L-deoxyribonucleosides and α-deoxyribonucleosides. In some embodiments, immune stimulatory oligonucleotides according to the invention incorporate 3′ modifications, and further include nucleosides having unnatural internucleoside linkage positions, including, without limitation, 2′-5′,2′-2′,3′-3′ and 5′-5′ linkages.

In some embodiments, immune stimulatory oligoncucleotides according to the invention further include nucleosides having modified heterocyclic bases, including, without limitation, 5-hydroxycytosine, 5-hydroxymethylcytosine, 4-thiouracil, 6-thioguanine, 7-deazaguanine, inosine, nitropyrrole, C5-propynylpyrimidine, N4-alkylcytosine, such as N4-ethylcytosine, and diaminopurines, including, without limitation, 2,6-diaminopurine.

By way of specific illustration and not by way of limitation, for example, in the immune stimulatory domain of structure (III), a methylphosphonate internucleoside linkage at position N1 or Nn is an immune stimulatory moiety, a linker having a length of from about 2 angstroms to about 200 angstroms, C2-C18 alkyl linker at position X1 is an immune stimulatory moiety, and a β-L-deoxyribonucleoside at position X1 is an immune stimulatory moiety. See Table 1 below for representative positions and structures of immune stimulatory moieties. It is to be understood that reference to a linker as the immune stimulatory moiety at a specified position means that the nucleoside residue at that position is substituted at its 3′-hydroxyl with the indicated linker, thereby creating a modified internucleoside linkage between that nucleoside residue and the adjacent nucleoside on the 3′ side. Similarly, reference to a modified internucleoside linkage as the immune stimulatory moiety at a specified position means that the nucleoside residue at that position is linked to the adjacent nucleoside on the 3′ side by way of the recited linkage.

TABLE 1

| Position | TYPICAL IMMUNE STIMULATORY MOIETIES |
|---|---|
| N1 | Naturally-occurring nucleosides, abasic nucleoside, N³- |
| | methyl-dC, N¹-methyl-dG, arabinonucleoside, 2′-deoxyuridine, |
| | β-L-deoxyribonucleoside C2-C18 alkyl linker, poly(ethylene |
| | glycol) linkage, 2-aminobutyl-1,3-propanediol linker (amino |
| | linker), 2′-5′ internucleoside linkage, methylphosphonate |
| | internucleoside linkage |
| Nn | Naturally-occurring nucleosides, abasic nucleoside, N³-methyl- |
| | dC, N¹-methyl-dG, arabinonucleosides, 2′-deoxyuridine, 2′-O- |
| | substituted ribonucleoside, 2′-5′ internucleoside linkage, |
| | methylphosphonate internucleoside linkage, provided that N1 |
| | and N2 cannot both be abasic linkages |

Table 2 shows representative positions and structures of immune stimulatory moieties within an immune modulatory oligonucleotide having an upstream potentiation domain. As used herein, the term “Spacer 9” refers to a poly(ethylene glycol) linker of formula —O—(CH₂CH₂—O)ₙ—, wherein n is 3. The term “Spacer 18” refers to a poly(ethylene glycol) linker of formula —O—(CH₂CH₂—O)ₙ—, wherein n is 6. As used herein, the term “C2-C18 alkyl linker refers to a linker of formula —O—(CH₂)_{q}—O—, where q is an integer from 2 to 18. Accordingly, the terms “C3-linker” and “C3-alkyl linker” refer to a linker of formula —O—(CH₂)₃—O—, which may be substituted or unsubstituted, branched or unbranched (e.g. 1,2,3, propanetriol). For each of Spacer 9, Spacer 18, and C2-C18 alkyl linker, the linker is connected to the adjacent nucleosides by way of phosphodiester, phosphorothioate, or phosphorodithioate linkages.

TABLE 2

| Position | TYPICAL IMMUNE STIMULATORY MOIETY |
|---|---|
| 5′ N2 | Naturally-occurring nucleosides, 2-aminobutyl-1,3-propanediol |
| | linker |
| 5′ N1 | Naturally-occurring nucleosides, β-L-deoxyribonucleoside, |
| | C2-C18 alkyl linker, poly(ethylene glycol), abasic linker, |
| | 2-aminobutyl-1,3-propanediol linker |
| 3′ N1 | Naturally-occurring nucleosides, 1′,2′-dideoxyribose, 2′-O- |
| | methyl-ribonucleoside, C2-C18 alkyl linker, Spacer 9, Spacer 18 |
| 3′ N2 | Naturally-occurring nucleosides, 1′,2′-dideoxyribose, 3′- |
| | deoxyribonucleoside, β-L-deoxyribonucleoside, 2′-O-propargyl- |
| | ribonucleoside, C2-C18 alkyl linker, Spacer 9, Spacer 18, |
| | methylphosphonate internucleoside linkage |
| 3′ N 3 | Naturally-occurring nucleosides, 1′,2′-dideoxyribose, C2-C18 |
| | alkyl linker, Spacer 9, Spacer 18, methylphosphonate |
| | internucleoside linkage, 2′-5′ internucleoside linkage, |
| | d(G)n, polyI-polyC |
| 3′N 2 + | 1′,2′-dideoxyribose, β-L-deoxyribonucleoside, C2-C18 alkyl |
| 3′N 3 | linker, d(G)n, polyI-polyC |
| 3′N3 + | 2′-O-methoxyethyl-ribonucleoside, methylphosphonate |
| 3′ N 4 | internucleoside linkage, d(G)n, polyI-polyC |
| 3′N5 + | 1′,2′-dideoxyribose, C2-C18 alkyl linker, d(G)n, polyI-polyC |
| 3′ N 6 | |
| 5′N1 + | 1′,2′-dideoxyribose, d(G)n, polyI-polyC |
| 3′ N 3 | |

Table 3 shows representative positions and structures of immune stimulatory moieties within an immune modulatory oligonucleotide having a downstream potentiation domain.

TABLE 3

| Position | TYPICAL IMMUNE STIMULATORY MOIETY |
|---|---|
| 5′ N2 | methylphosphonate internucleoside linkage |
| 5′ N1 | methylphosphonate internucleoside linkage |
| 3′ N1 | 1′,2′-dideoxyribose, methylphosphonate internucleoside linkage, |
| | 2′-O-methyl |
| 3′ N2 | 1′,2′-dideoxyribose, β-L-deoxyribonucleoside, C2-C18 alkyl |
| | linker, Spacer 9, Spacer 18, 2-aminobutyl-1,3-propanediol |
| | linker, methylphosphonate internucleoside linkage, 2′-O-methyl |
| 3′ N3 | 3′-deoxyribonucleoside, 3′-O-substituted ribonucleoside, 2′- |
| | O-propargyl-ribonucleoside |
| 3′N2 + | 1′,2′-dideoxyribose, β-L-deoxyribonucleoside |
| 3′ N3 | |

The immune modulatory oligonucleotides according to the invention comprise at least two oligonucleotides linked at their 3′ ends or internucleoside linkage or a functionalized nucleobase or sugar via a non-nucleotidic linker. For purposes of the invention, a “non-nucleotidic linker” is any moiety that can be linked to the oligonucleotides by way of covalent or non-covalent linkages. Such linker is from about 2 angstroms to about 200 angstroms in length. Several examples of linkers are set forth below. Non-covalent linkages include, but are not limited to, electrostatic interaction, hydrophobic interactions, π-stacking interactions, and hydrogen bonding. The term “non-nucleotidic linker” is not meant to refer to an internucleoside linkage, as described above, e.g., a phosphodiester, phosphorothioate, or phosphorodithioate functional group, that directly connects the 3′-hydroxyl groups of two nucleosides. For purposes of this invention, such a direct 3′-3′ linkage (no linker involved) is considered to be a “nucleotidic linkage.”

In some embodiments, the non-nucleotidic linker is a metal, including, without limitation, gold particles. In some other embodiments, the non-nucleotidic linker is a soluble or insoluble biodegradable polymer bead.

In yet other embodiments, the non-nucleotidic linker is an organic moiety having functional groups that permit attachment to the oligonucleotide. Such attachment is by any stable covalent linkage. As a non-limiting example, the linker may be attached to any suitable position on the nucleoside. In some embodiments, the linker is attached to the 3′-hydroxyl. In such embodiments, the linker comprises a hydroxyl functional group, which is attached to the 3′-hydroxyl by means of a phosphodiester, phosphorothioate, phosphorodithioate or non-phosphate-based linkages.

In some embodiments, the non-nucleotidic linker is a biomolecule, including, without limitation, polypeptides, antibodies, lipids, antigens, allergens, and oligosaccharides. In some other embodiments, the non-nucleotidic linker is a small molecule. For purposes of the invention, a small molecule is an organic moiety having a molecular weight of less than 1,000 Da. In some embodiments, the small molecule has a molecular weight of less than 750 Da.

In some embodiments, the small molecule is an aliphatic or aromatic hydrocarbon, either of which optionally can include, either in the linear chain connecting the oligonucleotides or appended to it, one or more functional groups selected from the group consisting of hydroxy, amino, thiol, thioether, ether, amide, thioamide, ester, urea, and thiourea. The small molecule can be cyclic or acyclic. Examples of small molecule linkers include, but are not limited to, amino acids, carbohydrates, cyclodextrins, adamantane, cholesterol, haptens, and antibiotics. However, for purposes of describing the non-nucleotidic linker, the term “small molecule” is not intended to include a nucleoside.

In some embodiments, the small molecule linker is glycerol or a glycerol homolog of the formula HO—(CH₂)ₙ—CH(OH)—(CH₂)ₚ—OH, wherein o and p independently are integers from 1 to about 6, from 1 to about 4, or from 1 to about 3. In some other embodiments, the small molecule linker is a derivative of 1,3-diamino-2-hydroxypropane. Some such derivatives have the formula HO—(CH₂)ₘ—C(O)NH—CH₂—CH(OH)—CH₂—NHC(O)—(CH₂)ₘ—OH, wherein m is an integer from 0 to about 10, from 0 to about 6, from 2 to about 6, or from 2 to about 4.

Some non-nucleotidic linkers according to the invention permit attachment of more than two oligonucleotides. For example, the small molecule linker glycerol has three hydroxyl groups to which oligonucleotides may be covalently attached. Some immune modulatory oligonucleotides according to the invention, therefore, comprise more than two oligonucleotides linked at their 3′ ends to a non-nucleotidic linker.

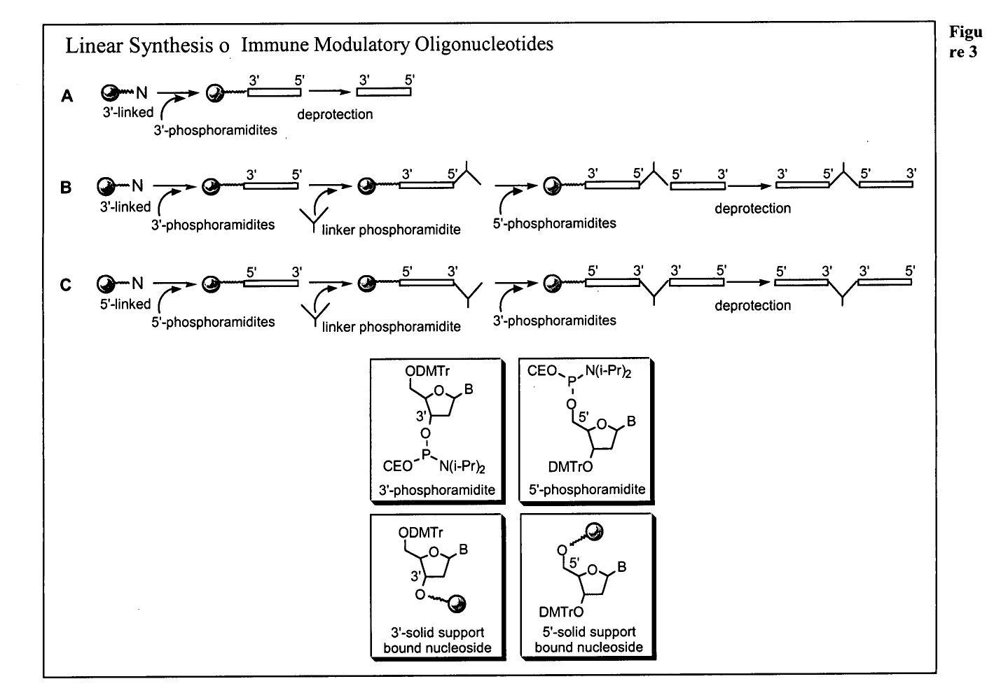

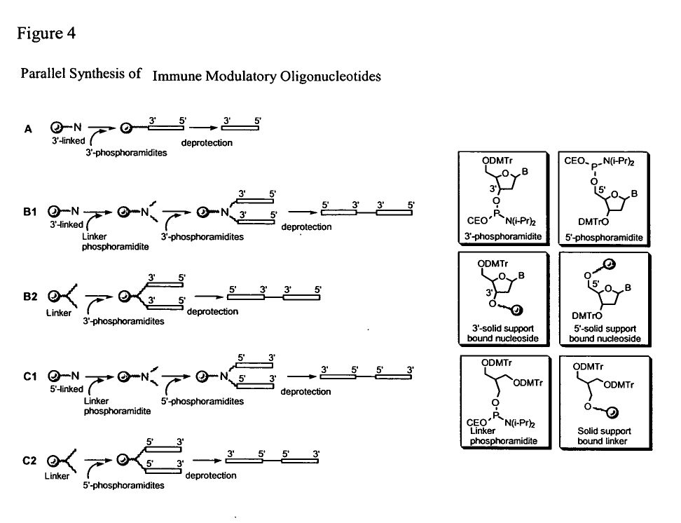

The immune modulatory oligonucleotides of the invention may conveniently be synthesized using an automated synthesizer and phosphoramidite approach as schematically depicted in FIGS. 3 and 4, and further described in the Examples. In some embodiments, the immune modulatory oligonucleotides are synthesized by a linear synthesis approach (see FIG. 3). As used herein, the term “linear synthesis” refers to a synthesis that starts at one end of the immune modulatory oligonucleotide and progresses linearly to the other end. Linear synthesis permits incorporation of either identical or un-identical (in terms of length, base composition and/or chemical modifications incorporated) monomeric units into the immune modulatory oligonucleotides.

An alternative mode of synthesis is “parallel synthesis”, in which synthesis proceeds outward from a central linker moiety (see FIG. 4). A solid support attached linker can be used for parallel synthesis, as is described in U.S. Pat. No. 5,912,332. Alternatively, a universal solid support (such as phosphate attached controlled pore glass) support can be used.

Parallel synthesis of immune modulatory oligonucleotides has several advantages over linear synthesis: (1) parallel synthesis permits the incorporation of identical monomeric units; (2) unlike in linear synthesis, both (or all) the monomeric units are synthesized at the same time, thereby the number of synthetic steps and the time required for the synthesis is the same as that of a monomeric unit; and (3) the reduction in synthetic steps improves purity and yield of the final immune modulatory oligonucleotide product.

At the end of the synthesis by either linear synthesis or parallel synthesis protocols, the immune modulatory oligonucleotides may conveniently be deprotected with concentrated ammonia solution or as recommended by the phosphoramidite supplier, if a modified nucleoside is incorporated. The product immune modulatory oligonucleotide can be purified by reversed phase HPLC, detritylated, desalted and dialyzed.

Table 4 shows representative immune modulatory oligonucleotides according to the invention.

TABLE 4A

Examples of Immune Modulatory
Oligonucleotides Sequences

| | |
|---|---|
| SEQ | |
| ID NO. | Sequences and Modification |

| | |
|---|---|
| 1 | 5′-CTATCTGAC₁GTTCTCTGT-3′ |
| 2 | 5′-CTATCTGACG₁TTCTCTGT-3′ |
| 3 | 5′-CTATCTGTC₁GTTCTGTGT-3′ |
| 4 | 5′-CTATCTGTCG₁TTCTCTGT-3′ |
| 5 | 5′-CTATCTGAGC₁TTCTCTGT-3′ |
| 6 | 5′-CTATCTGAG₁CTTCTCTGT-3′ |
| 7 | 5′-TCTGAC₁GTTCT-X-TCTTGC₁AGTCT-5′ |
| 8 | 5′-TCTGACG₁TTCT-X-TCTTG₁CAGTCT-5′ |
| 9 | 5′-TCTGTC₁GTTCT-X-TCTTGC₁TGTCT-5′ |
| 10 | 5′-TCTGTCG₁TTCT-X-TCTTG₁CTGTCT-5′ |
| 11 | 5′-TCTGAGC₁TTCT-X-TCTTC₁GAGTCT-5′ |
| 12 | 5′-TCTGAG₁CTTCT-X-TCTTCG₁AGTCT-5′ |
| 13 | 5′-CTATCTGACGTTCTCTGT-3′ |
| 14 | 5′-CTATCTGTCGTTCTCTGT-3′ |
| 15 | 5′-CTATCTCACCTTCTCTG-3′ (control) |
| 16 | 5′-TCTGACGTTCT-X-TCTTGCAGTCT-5′ |
| 17 | 5′-TCTGACG₂TTCT-X-TCTTG₂CAGTCT-5′ |
| 18 | 5′-TCTCACCTTCT-X-TCTTCCACTCT-5′ (control) |
| 19 | 5′-ACACACCAACT-X-TCAACCACACA-5′ (control) |
| 20 | 5′-TCTGTCG₂TTCT-X-TCTTG₂CTGTCT-5′ |
| 21 | 5′-TCTGACGTTCT-X-TCTTGCAGTCT-5′ |
| 22 | 5′-TCTGAC₂GTTCT-X-TCTTGC₂AGTCT-5′ |
| 23 | 5′-TCTGAC₃GTTCT-X-TCTTGC₃AGTCT-5′ |
| 24 | 5′-TCTGAGC₂TTCT-X-TCTTC₂GAGTCT-5′ (control) |
| 25 | 5′-TCTGAGC₃TTCT-X-TCTTC₃GAGTCT-5′ (control) |
| 26 | 5′-TCTGTCGTTCT-X-TCTTGCTGTCT-5′ |
| 27 | 5′-TCTGTC₃GTTCT-X-TCTTGC₃TGTCT-5′ |
| 28 | 5′-TCTGTC₂GTTCT-X-TCTTGC₂TGTCT-5′ |
| 29 | 5′-ACACACCAACT-X-TCAACCACACA-5′ (control) |
| 30 | 5′-TC₃G₂AAC₃G₃TTC₃G₃-X-G₂C₃TTG₃C₃AAG₂C₃T-5′ |
| 31 | 5′-TC₄G₂AAC₄G₃TTC₄G₂-X-G₂C₄TTG₃C₄AAG₂C₄T-5′ |
| 32 | 5′-TC₃G₂AAC₃G₂TTCG₂-Y-TCTTG₃C₃TGTCT-5′ |
| 33 | 5′-TC₄G₂AAC₄G₂TTC₄G₂-Y-TCTTG₃C₄TGTCT-5′ |

C₁ = N³-methyl-dC;
C₂ = dF;
C₃ = ψ-iso-dC;
C₄ = 1-(2′-deoxy-β-D-ribofuranosyl)-2-oxo-7-deaza-8-methylpurine;
G₁ = N¹-methyl-dG;
G₂ = 7-deaza-dG;
G₃ = Arabinoguanosine;
X = Glycerol linker;
Y = C3 linker

Certain embodiments of this aspect of the invention provides immune modulatory oligonucleotide conjugates comprising an immune stimulatory oligonucleotide, as described above, and a compound conjugated to the immune stimulatory oligonucleotide at a position other than the accessible 5′ end. In some embodiments, the compound is conjugated to the non-nucleotidic linker. In some other embodiments, the compound is conjugated to the oligonucleotide at a position other than its 5′ end. Suitable compounds which can be conjugated to the immune modulatory oligonucleotides of the invention include, but are not limited to, cholesterol, different lengths of polyethylene glycol, peptides, antibodies, proteins, vaccines, lipids, antigens, and any immune stimulatory small molecule such as, but not limited to, imiquimod, R848, loxoribine, isatorbin as well as chemotherapeutic agents.

The antigen includes, but is not limited to, antigens associated with a pathogen, antigens associated with a cancer, antigens associated with an auto-immune disorder, and antigens associated with other diseases such as, but not limited to, veterinary or pediatric diseases. In some embodiments, the antigen produces a vaccine effect. For purposes of the invention, the term “associated with” means that the antigen is present when the pathogen, cancer, auto-immune disorder, food allergy, respiratory allergy, asthma or other disease is present, but either is not present, or is present in reduced amounts, when the pathogen, cancer, auto-immune disorder, food allergy, respiratory allergy, or disease is absent.

The immune stimulatory oligonucleotide is covalently linked to the antigen, or it is otherwise operatively associated with the antigen. As used herein, the term “operatively associated with” refers to any association that maintains the activity of both immune stimulatory oligonucleotide and antigen. Non-limiting examples of such operative associations include being part of the same liposome or other such delivery vehicle or reagent. In embodiments wherein the immune stimulatory oligonucleotide is covalently linked to the antigen, such covalent linkage preferably is at any position on the immune stimulatory oligonucleotide other than an accessible 5′ end of an immune stimulatory oligonucleotide. For example, the antigen may be attached at an internucleoside linkage or may be attached to the non-nucleotidic linker. Alternatively, the antigen may itself be the non-nucleotidic linker.

In a second aspect, the invention provides pharmaceutical formulations comprising an immune modulatory oligonucleotide or immune modulatory oligonucleotide conjugate according to the invention and a physiologically acceptable carrier. As used herein, the term “physiologically acceptable” refers to a material that does not interfere with the effectiveness of the immune modulatory oligonucleotide and is compatible with a biological system such as a cell, cell culture, tissue, or organism. Preferably, the biological system is a living organism, such as a vertebrate.

As used herein, the term “carrier” encompasses any excipient, diluent, filler, salt, buffer, stabilizer, solubilizer, lipid, or other material well known in the art for use in pharmaceutical formulations. It will be understood that the characteristics of the carrier, excipient, or diluent will depend on the route of administration for a particular application. The preparation of pharmaceutically acceptable formulations containing these materials is described in, e.g., Remington's Pharmaceutical Sciences, 18th Edition, ed. A. Gennaro, Mack Publishing Co., Easton, Pa., 1990.

In a third aspect, the invention provides methods for generating an immune response in a vertebrate, such methods comprising administering to the vertebrate an immune modulatory oligonucleotide or immune modulatory oligonucleotide conjugate according to the invention. In some embodiments, the vertebrate is a mammal. For purposes of this invention, the term “mammal” is expressly intended to include humans. In certain embodiments, the immune modulatory oligonucleotide or immune modulatory oligonucleotide conjugate is administered to a vertebrate in need of immune stimulation.

In the methods according to this aspect of the invention, administration of immune modulatory oligonucleotide or immune modulatory oligonucleotide conjugate can be by any suitable route, including, without limitation, parenteral, oral, sublingual, transdermal, topical, mucosal, inhalation, intranasal, aerosol, intraocular, intratracheal, intrarectal, vaginal, by gene gun, dermal patch or in eye drop or mouthwash form. Administration of the therapeutic compositions of immune modulatory oligonucleotides can be carried out using known procedures at dosages and for periods of time effective to reduce symptoms or surrogate markers of the disease. When administered systemically, the therapeutic composition is preferably administered at a sufficient dosage to attain a blood level of immune modulatory oligonucleotide from about 0.0001 micromolar to about 10 micromolar. For localized administration, much lower concentrations than this may be effective, and much higher concentrations may be tolerated. Preferably, a total dosage of immune modulatory oligonucleotide ranges from about 0.001 mg per patient per day to about 200 mg per kg body weight per day. It may be desirable to administer simultaneously, or sequentially a therapeutically effective amount of one or more of the therapeutic compositions of the invention to an individual as a single treatment episode.

In certain embodiments, immune modulatory oligonucleotide or immune modulatory oligonucleotide conjugate according to the invention are administered in combination with vaccines, antibodies, cytotoxic agents, allergens, antibiotics, antisense oligonucleotides, peptides, proteins, gene therapy vectors, DNA vaccines and/or adjuvants to enhance the specificity or magnitude of the immune response. In these embodiments, the immune modulatory oligonucleotides of the invention can variously act as adjuvants and/or produce direct immune stimulatory effects.

Either the immune modulatory oligonucleotide or immune modulatory oligonucleotide conjugate or the vaccine, or both, may optionally be linked to an immunogenic protein, such as keyhole limpet hemocyanin (KLH), cholera toxin B subunit, or any other immunogenic carrier protein. Any of the plethora of adjuvants may be used including, without limitation, Freund's complete adjuvant, KLH, monophosphoryl lipid A (MPL), alum, and saponins, including QS-21, imiquimod, R848, or combinations thereof.

For purposes of this aspect of the invention, the term “in combination with” means in the course of treating the same disease in the same patient, and includes administering the immune modulatory oligonucleotide and/or the vaccine and/or the adjuvant in any order, including simultaneous administration, as well as temporally spaced order of up to several days apart. Such combination treatment may also include more than a single administration of the immune modulatory oligonucleotide, and/or independently the vaccine, and/or independently the adjuvant. The administration of the immune modulatory oligonucleotide and/or vaccine and/or adjuvant may be by the same or different routes.

The methods according to this aspect of the invention are useful for model studies of the immune system. The methods are also useful for the prophylactic or therapeutic treatment of human or animal disease. For example, the methods are useful for pediatric and veterinary vaccine applications.

In a fourth aspect, the invention provides methods for therapeutically treating a patient having a disease or disorder, such methods comprising administering to the patient an immune modulatory oligonucleotide or immune modulatory oligonucleotide conjugate according to the invention. In various embodiments, the disease or disorder to be treated is cancer, an autoimmune disorder, airway inflammation, inflammatory disorders, allergy, asthma or a disease caused by a pathogen. Pathogens include bacteria, parasites, fingi, viruses, viroids and prions. Administration is carried out as described for the third aspect of the invention.

For purposes of the invention, the term “allergy” includes, without limitation, food allergies and respiratory allergies. The term “airway inflammation” includes, without limitation, asthma. As used herein, the term “autoimmune disorder” refers to disorders in which “self” proteins undergo attack by the immune system. Such term includes autoimmune asthma.

In a fifth aspect, the invention provides methods for preventing a disease or disorder, such methods comprising administering to the patient an immune modulatory oligonucleotide or immune modulatory oligonucleotide conjugate according to the invention. In various embodiments, the disease or disorder to be prevented is cancer, an autoimmune disorder, airway inflammation, inflammatory disorders, allergy, asthma or a disease caused by a pathogen. Pathogens include bacteria, parasites, fungi, viruses, viroids, and prions. Administration is carried out as described for the third aspect of the invention.

In any of the methods according to this aspect of the invention, the immune modulatory oligonucleotide or immune modulatory oligonucleotide conjugate can be administered in combination with any other agent useful for treating the disease or condition that does not diminish the immune stimulatory effect of the immune modulatory oligonucleotide. In any of the methods according to the invention, the agent useful for treating the disease or condition includes, but is not limited to, vaccines, antigens, antibodies, cytotoxic agents, allergens, antibiotics, antisense oligonucleotides, peptides, proteins, gene therapy vectors, DNA vaccines and/or adjuvants to enhance the specificity or magnitude of the immune response, or co-stimulatory molecules such as cytokines, chemokines, protein ligands, trans-activating factors, peptides and peptides comprising modified amino acids. For example, in the treatment of cancer, it is contemplated that the immune modulatory oligonucleotide or immune modulatory oligonucleotide conjugate may be administered in combination with a chemotherapeutic compound or a monoclonal antibody. Alternatively, the agent can include DNA vectors encoding for antigen or allergen. In these embodiments, the immune modulatory oligonucleotides of the invention can variously act as adjuvants and/or produce direct immune modulatory effects.

Chemotherapeutic agents used in the method according to the invention include, without limitation Gemcitabine, methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MM1270, BAY 12-9566, RAS farnesyl transferase inhibitor, farnesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, IS1641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Placlitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT (Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP 16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26), Vindesine sulfate, tyrosine kinase inhibitors, such as EGFR and VEGF inhibitors including, but not limited to, Lapatinib (EGFR and ErbB-2 (Her2/neu) dual tyrosine kinase inhibitor (GSK)), Gefitinib (ZD1839/Iressa (AstraZeneca)), Erlotinib (Tarceva—EGFR/HER1 inhibitor (Genentech)), Thalidomide ((Thalidomide)-anti-angeogenic drug), Imatinib (Glivec) and Vatalanib (VEGFR tyrosine kinase inhibitor), Sorafenib (Raf kinase inhibitor (Bayer)), VX-680 (Aurora kinase inhibitor), Sutent (Receptor Tyrosine Kinases (RTKs) inhibitor (Pfizer)), Bortezomib ((Velcade) proteosome inhibitor), Temozolomide ((Temodal) alkylating agent), and Interferon alpha (Intron A, Roferon A).

Passive immunotherapy in the form of antibodies, and particularly monoclonal antibodies, has been the subject of considerable research and development as anti-cancer agents. The term “monoclonal antibody” as used herein refers to an antibody molecule of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. Examples of anti-cancer agents include, but are not limited to, Panorex (Glaxo-Welicome), Rituxan (IDEC/Genentech/Hoffman la Roche), Mylotarg (Wyeth), Campath (Millennium), Zevalin (IDEC and Schering AG), Bexxar (Corixa/GSK), Erbitux (Imclone/BMS), Avastin (Genentech), Herceptin (Genentech/Hoffman la Roche), Cetuximab (Imclone) and Panitumumab (Abgenix/Amgen). Antibodies may also be employed in active immunotherapy utilizing anti-idiotype antibodies which appear to mimic (in an immunological sense) cancer antigens. Monoclonal antibodies can be generated by methods known to those skilled in the art of recombinant DNA technology.

The examples below are intended to further illustrate certain embodiments of the invention, and are not intended to limit the scope of the invention.

### EXAMPLES

### Example 1

### Synthesis of Oligonucleotides Containing Immune Stimulatory Moieties

Oligonucleotides were synthesized on a 1 μmol to 0.1 mM scale using an automated DNA synthesizer (OligoPilot II, AKTA, (Amersham) and/or Expedite 8909 (Applied Biosystem)), following the linear synthesis or parallel synthesis procedures outlined in FIGS. 3 and 4.

5′-DMT dA, dG, dC and T phosphoramidites were purchased from Proligo (Boulder, Colo.). 5′-DMT 7-deaza-dG and araG phosphoramidites were obtained from Chemgenes (Wilmington, Mass.). DiDMT-glycerol linker solid support was obtained from Chemgenes. 1-(2′-deoxy-β-D-ribofuranosyl)-2-oxo-7-deaza-8-methyl-purine amidite was obtained from Glen Research (Sterling, Va.), 2′-O-methylribonuncleoside amidites were obtained from Promega (Obispo, Calif.). All oligonucleotides were phosphorothioate backbone modified.

All nucleoside phosphoramidites were characterized by ³¹P and ¹H NMR spectra. Modified nucleosides were incorporated at specific sites using normal coupling cycles recommended by the supplier. After synthesis, oligonucleotides were deprotected using concentrated ammonium hydroxide and purified by reverse phase HPLC, detritylation, followed by dialysis. Purified oligonucleotides as sodium salt form were lyophilized prior to use. Purity was tested by CGE and MALDI-TOF MS. Endotoxin levels were determined by LAL test and were below 1.0 EU/mg.

### Example 2

### Mouse Spleen Cell Cultures

Four-to-eight-week-old C57BL/6 and BALB/c mice were obtained from Taconic Farms, Germantown, N.Y. and maintained in accordance with Idera's IACUC-approved animal protocols. All the animal studies reported in the paper were carried out following Idera's IACUC guidelines and approved protocols. Spleen cells from 4-8 week old BALB/c or C57BL/6 mice were prepared and cultured in RPMI complete medium. Mouse spleen cells were plated in 24-well dishes at 5×10⁶ cells/ml. IMOs dissolved in TE buffer (10 mM Tris-HCL, pH 7.5, 1 mM EDTA) were added to a final concentration of 0.03, 0.1, 0.3, 1.0, 3.0 or 10 μg/ml to the cell cultures. The cells were then incubated at 37° C. for 24 hr and the supernatants were collected for ELISA assays.

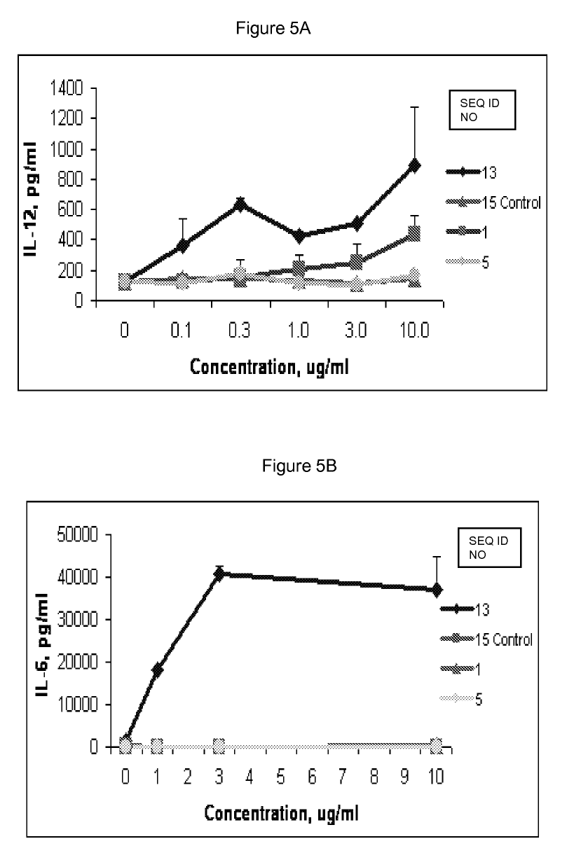

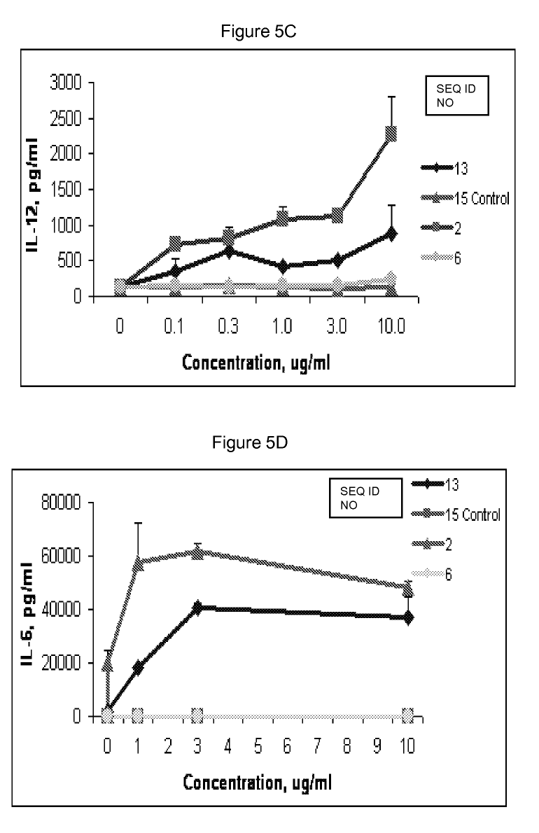

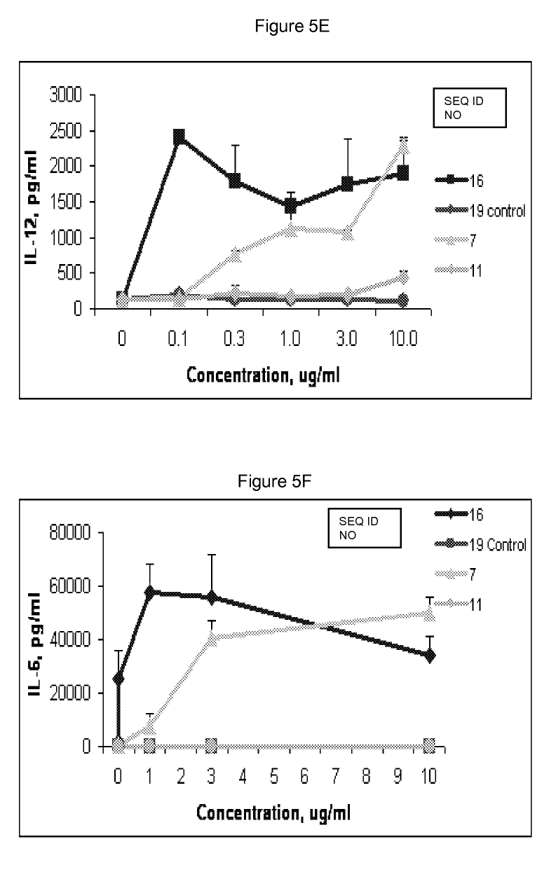

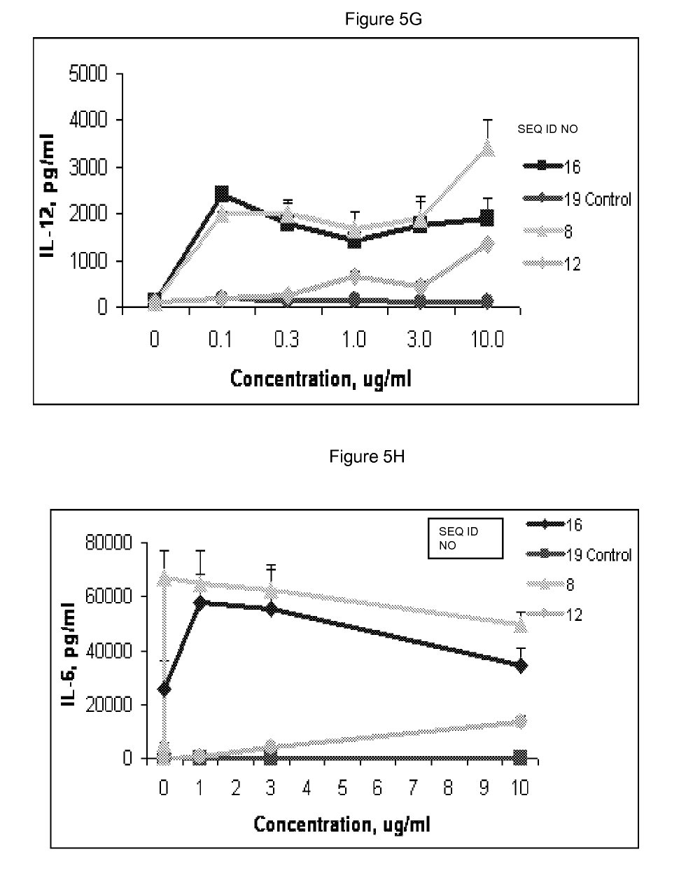

IL-12 and IL-6 levels in supernatants were measured by sandwich ELISA. The results are shown in FIGS. 5A through 5D. The required reagents including cytokine antibodies and standards were purchased from BD Pharmingen. Streptavidin-Peroxidase and substrate were from KPL.

### Example 3

### Human PBMC Isolation

Peripheral blood mononuclear cells (PBMCS) from freshly drawn healthy volunteer blood (CBR Laboratories, Boston, Mass.) were isolated by Ficoll density gradient centrifugation method (Histopaque-1077, Sigma).

### Example 4

### Cytokine ELISAs

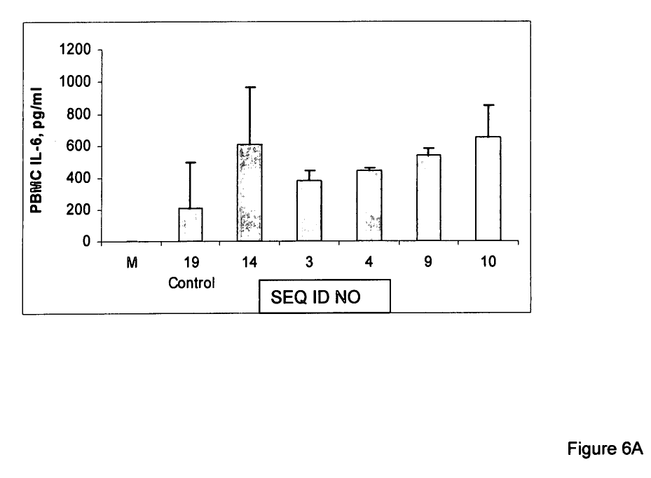

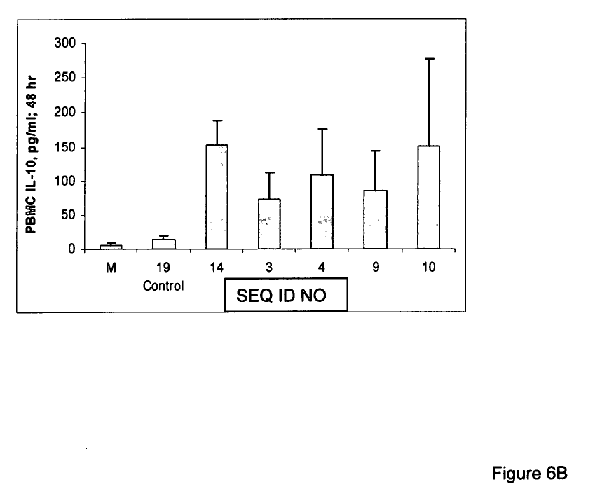

Human PBMCs were plated in 48-well plates using 5×10⁶ cells/ml. The IMOs dissolved in DPBS (pH 7.4; Mediatech) were added to a final concentration of 10.0 μg/ml to the cell cultures. The cells were then incubated at 37° C. for 24 hr and the supernatants were collected for ELISA assays. The experiments were performed in triplicate wells. The levels of IL-6 and IL-10 were measured by sandwich ELISA. The results are shown in FIGS. 6A and 6B. The required reagents, including cytokine antibodies and standards, were purchased from PharMingen.

### Example 5

### HEK293 Cell Cultures

HEK293/mTLR9 cells (Invivogen, San Diego, Calif.) were cultured in 48-well plates in 250 μl/well DMEM supplemented with 10% heat-inactivated FBS in a 5% CO₂ incubator.

### Example 6

### Reporter Gene Transformation

At 80% confluence, cultures were transiently transformed with 400 ng/ml of Seap reporter plasmid (pNifty2-Seap) (San Diego Calif.) in the presence of 4 μl/ml of Lipofectamine (Invitrogen, CA) in culture medium. Plasmid DNA and Lipofectamine were diluted separately in serum-free medium and incubated at room temperature for 5 minutes. After incubation, the diluted DNA and Lipofectamine were mixed and the mixtures were incubated at room temperature for 20 minutes. 25 μl of the DNA/Lipofectamine mixture containing 100 ng plasmid DNA and 1 μl of Lipofectamine was added to each well of the cell culture plate, and the cultures were continued for 4 hours.

### Example 7

### Immune Modulatory Oligonucleotide Treatment

After transfection, medium was replaced with fresh culture medium, and stimulating oligos, immune modulatory oligonucleotides, were individually added to the cultures and the cultures were continued for 18 hours.

### Example 8

### SEAP Assay

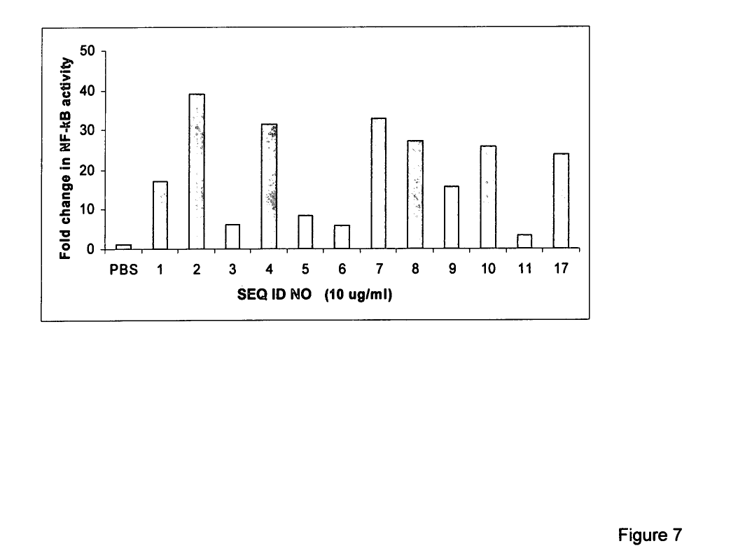

At the end of oligo, immune modulatory oligonucleotide, treatment, 30 μl of culture supernatant was taken from each treatment and used for SEAP assay. Manufacturer's protocol (Invivogen) was followed for the assay. Signals were detected by a plate reader at 405 nm. The results are shown in FIG. 7 and demonstrate that administration of immune modulatory oligonucleotides containing novel bases generates unique TLR9 activation profiles.

### Example 9

### Assessment of Mouse Serum Cytokine Levels

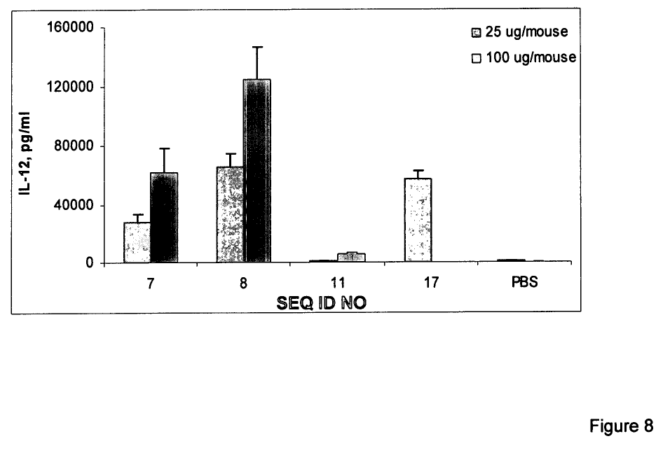

Female C57BL/6 mice, 5-6 weeks old, were obtained from Taconic Farms, Germantown, N.Y. and maintained in accordance with Idera Pharmaceutical's IACUC approved animal protocols. Mice (n=2-3) were injected subcutaneously (s.c) with individual immune modulatory oligonucleotides at 25 or 100 μg dose or 1 mg/kg (single dose). Serum was collected by retro-orbital bleeding 4 hr after immune modulatory oligonucleotide administration and IL-12 was determined by sandwich ELISA. The results are shown in FIG. 8 and demonstrate that administration in vivo of immune modulatory oligonucleotides containing novel bases generates unique IL-12 profiles. All reagents, including cytokine antibodies and standards were purchased from PharMingen. (San Diego, Calif.).

### Example 10

### Mouse Spleen Cell Cultures

Spleen cells from C57BL/6 mice were prepared and cultured in RPMI complete medium consisting of RPMI 1640 with 10% fetal calf serum (FCS), 100 U/ml penicillin, 100 μg/ml streptomycin and 2 mM L-glutamine (HyClone, Logan, Utah). Mouse spleen cells were plated in 24-well plates at 5×10⁶ cells/ml. Individual immune modulatory oligonucleotides dissolved in TE buffer [10 mM Tris-HCl (pH 7.5) and 1 mM EDTA] were added to a final concentration of 3 or 10 μg/ml to the cell cultures. The cells were then incubated at 37° C. for 24 h and the supernatants were collected for cytokine analysis by enzyme-linked immunosorbent assays (ELISAs).

IL-12 and IL-6 levels in supernatants were measured by sandwich ELISA. The required reagents, including cytokine antibodies and standards, were purchased from BD Pharmingen (San Diego, Calif.). Streptavidin-peroxidase and TMB substrate were from Sigma (St. Louis, Mo.) and KPL (Gaithersburg, Md.), respectively.

### Example 11

### Human B-Cell Proliferation Assay

About 1×10⁵ B-cells purified from human PBMCs were stimulated with different concentrations of immune modulatory oligonucleotides for 64 h, then pulsed with 0.75 μCi of [³H]-thymidine and harvested 8 h later. The incorporation of [³H]-thymidine was measured by scintillation counter and the data are presented as counts per minute (c.p.m.).

### Example 12

### Human Multiplex Cytokine ELISAs

Human PBMCs were plated in 96-well plates at a concentration of 5×10⁶ cells/ml. The immune modulatory oligonucleotides dissolved in phosphate-buffered saline (PBS) were added to the cell cultures at a final concentration of 10 μg/ml. The cells were then incubated at 37° C. for 24 h. The supernatants were then analyzed for the listed cytokines using the Luminex-multiplex ELISA system. The human multiplex kit was obtained from invitrogen.

### Example 13

### Mouse Splenomegaly Assay

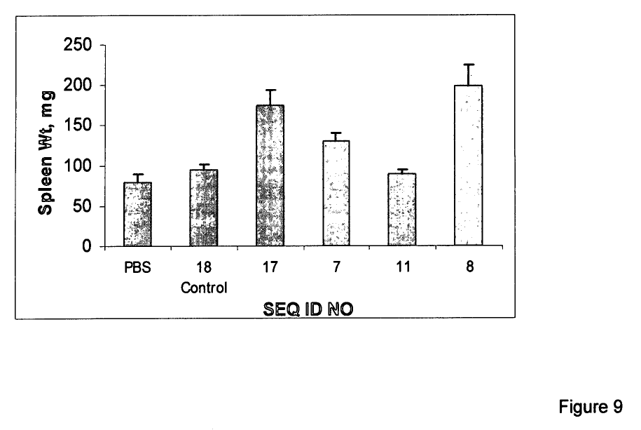

Female BALB/c mice (4-6 weeks, 19-21 gm) were divided into groups of three mice. immune modulatory oligonucleotides DNAs were dissolved in sterile PBS and administered subcutaneously (SC) to mice at a dose of 5 mg/kg. After 72 hrs, mice were sacrificed and the spleens were harvested and weighed. The results are shown in FIG. 9 and demonstrate that administration in vivo of immune modulatory oligonucleotides containing novel bases generates unique immune response profiles.

### Example 14

### OVA-Sensitized Mouse Spleen Cell Culture Assays

Four to six week old BALB/c female mice were obtained from Taconic (Germantown, N.Y.). The mice were given intraperitoneal injections of 20 μg of chicken ovalbumin (OVA; Sigma) in 100 μL of PBS mixed with 100 μL of ImjectAlum adjuvant (Pierce) on days 0, 7, and intranasally challenged on days 14, and 21 with 10 μg of OVA in 40 μl PBS. The mice were sacrificed 72 hr after the last challenge by CO₂ inhalation.

Spleens were excised and single cell suspensions were prepared as described above. Spleen cells were treated with immune modulatory oligonucleotides at different concentrations for 2 hr followed by treatment with 100 μg/mL of OVA.

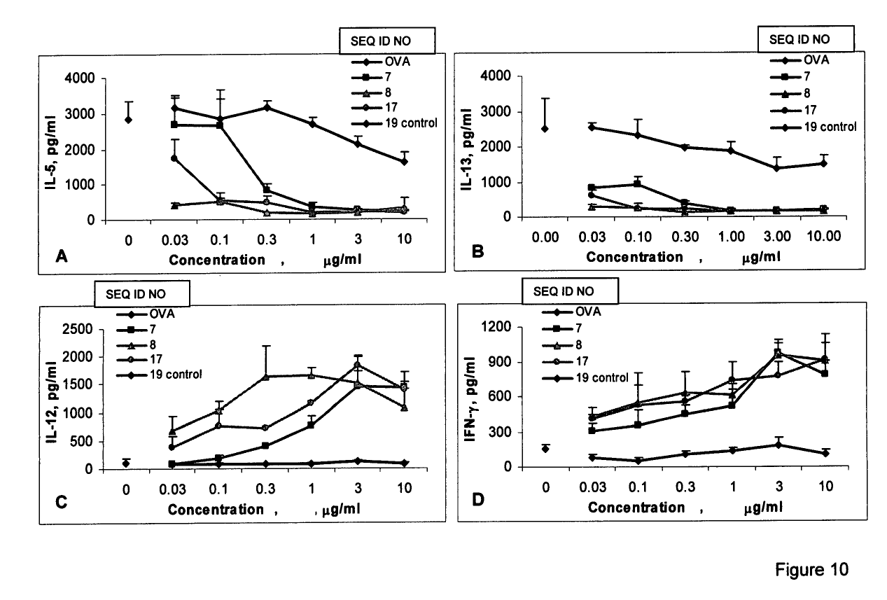

After 72 hr supernatants were collected and IL-5, IL-13, IL-12, and IFN-α levels were measured by ELISA as described above. The results are shown in FIGS. 10A-10D and demonstrate that administration of immune modulatory oligonucleotides containing novel bases generates unique cytokine/chemokine profiles, even in the presence of an immune system activator (e.g. ovalbumin), which vary with the base composition and the amount of the oligonucleotide administered.

### Example 15

### In Vivo Anti-Cancer Activity of Immune Modulatory Oligonucleotides in Combination with Chemotherapeutic Agents

PC3 cells can be cultured in 90% Ham's, F12K Medium with 10% Fetal Bovine Serum (FBS), in presence of 100 U/ml Penicillin and 100 μg/ml Streptomycin to establish the Human Prostate cancer model (PC3). Male athymic nude mice, 4-6 weeks old (Frederick Cancer Research and Development Center, Frederick, Md.), can be accommodated for 6 days for environmental adjustment prior to the study. Cultured PC3 cells can be harvested from the monolayer cultures, washed twice with Ham's, F12K Medium (10% FBS), resuspended in FBS-free Ham's, F12K Medium: Matrigel basement membrane matrix (Becton Dickinson Labware, Bedford, Mass.) (5:1; V/V), and injected subcutaneously (5×10⁶ cells, total volume 0.2 ml) into the left inguinal area of each of the mice. The animals can be monitored by general clinical observation, body weight, and tumor growth. Tumor growth can be monitored by the measurement, with calipers, of two perpendicular diameters of the implant. Tumor mass (weight in grams) can be calculated by the formula, ½a×b², where ‘a’ is the long diameter (cm) and ‘b’ is the short diameter (cm). When the mean tumor sizes reached ˜80 mg, the animals bearing human cancer xenografts can be randomly divided into the treatment and control groups (5 animals/group). The control group can receive sterile physiological saline (0.9% NaCl) only. Immune modulatory oligonucleotides of the invention, aseptically dissolved in physiological saline, can be administered by subcutaneously injection at dose of 0.5 or 1.0 mg/kg/day, 3 doses/week. A chemotherapeutic agent can be given twice by intraperitoneal injection at 160 mg/kg on Day 0 and 3.

### Example 16

### Synthesis of Oligonucleotides Containing Immune Modulatory Moieties

Immune modulatory oligonucleotides with 2′-deoxy-pyrido[2,3-d]pyrimidine-2,7(8H)-dione (dF) or 2′-deoxypseudoisocytidine (ψ-iso-dC) modifications were synthesized on a 2-μmol scale using β-cyanoethylphosphoramidite chemistry on a PerSeptive Biosystem 8909 Expedite DNA synthesizer. Di-DMT-protected glyceryl linker attached to CPG-solid-support was obtained from ChemGenes Corporation (Wilmington, Mass.). The 3′-phosphoramidites of dA, dG, dC, and T were obtained from Applied Biosystems, whereas, dmf-dG phosphoramidite was obtained from Glen Research (Sterling, Va.). Phosphoramidites of dF and W-iso-dC were obtained from Berry & Associates (Dexter, Mich.). Beaucage reagent was used as an oxidant to obtain the phosphorothioate backbone modification. Supplier recommended synthesis protocols were used for dF and ψ-iso-dC phosphoramidite incorporation and deprotection. After the synthesis, immune modulatory oligonucleotides were deprotected, purified by “trityl on” RP-HPLC, detritylated, and dialyzed against United States Pharmacopea-quality sterile water for irrigation (Braun, Irvine, Calif.). The immune modulatory oligonucleotides were lyophilized and dissolved again in distilled water and the concentrations were determined by measuring the UV absorbance at 260 nm. The purity of all the compounds synthesized was determined by denaturing PAGE and the sequence integrity was characterized by matrix-assisted laser desorption/ionization-time-of-flight (MALDI-TOF) mass spectrometry for molecular mass. All immune modulatory oligonucleotides (Table 4A) were synthesized and purified under identical conditions to minimize endotoxin contamination.

### Example 17

### Immune Modulatory Oligonucleotides Containing dF or ψ-iso-dC in CpG Motif Activate TLR9

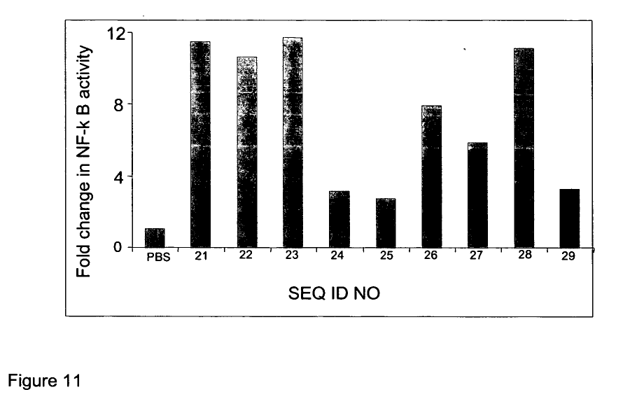

Activation of HEK293 cells expressing mouse TLR9 with immune modulatory oligonucleotides and control compounds at a concentration of 100 g/ml. The ability of immune modulatory oligonucleotides containing the dF or ψ-iso-dC modification to activate TLR9 was studied in HEK293 cells stably expressing mouse TLR9. Human secreted embryonic alkaline phosphatase (SEAP) gene is used as a NF-κB reporter. The results are presented as fold increase in NF-κB activation over PBS control (FIG. 11). immune modulatory oligonucleotides 27 and 28 (SEQ ID NO 27 and 28) (Table 4A), which contained dF or ψ-iso-dC, activated TLR9, as shown by an increase in NF-κB activity. These results demonstrate that dF or ψ-iso-dC modification is tolerated and functional in the C-position and further demonstrate that administration of immune modulatory oligonucleotides containing novel bases generates unique TLR9 activation profiles (FIG. 11).

### Example 18

### Immune Modulatory Oligonucleotides Containing dF or ψ-Iso-Dc in CpG Motif Induce Cytokine Secretion in Mouse Spleen Cell Cultures

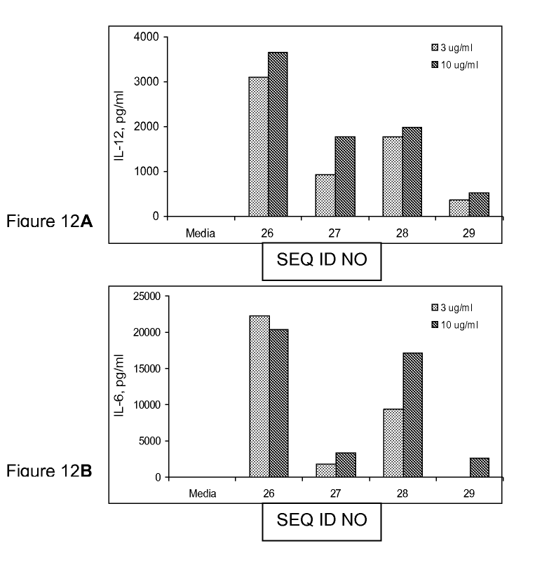

Induction of cytokine secretion by IMOs in C57BL/6 mouse spleen cell cultures. C57BL/6 mouse spleen cells were cultured in medium alone (M) or in the presence of immune modulatory oligonucleotides at various concentrations for 24 h and the levels of secreted IL-12 (FIG. 12A) and IL-6 (FIG. 12B) in culture supernatants were measured by ELISA. Data shown are at 3 and 10 μg/ml concentrations of immune modulatory oligonucleotides (FIGS. 12A and 12B). Immune modulatory oligonucleotides 27 (SEQ ID NO 27) and 28 (SEQ ID NO 28) containing dF or ψ-iso-dC induced IL-12 and IL-6 secretion in C57BL/6 mouse spleen cell cultures compared with control immune modulatory oligonucleotide 29 (SEQ ID NO 29) (FIGS. 12A and 12B). These results demonstrate that immune modulatory oligonucleotides with dF or ψ-iso-dC modifications are tolerated by and active on immune cells and further that administration of immune modulatory oligonucleotides containing novel bases generates unique IL-6 and IL-12 profiles, which vary with the base composition and the amount of the oligonucleotide administered.

### Example 19

### Immune Modulatory Oligonucleotides Containing dF or ψ-Iso-Dc in CpG Motif Induce Splenomegaly and Cytokines In Vivo in Mice

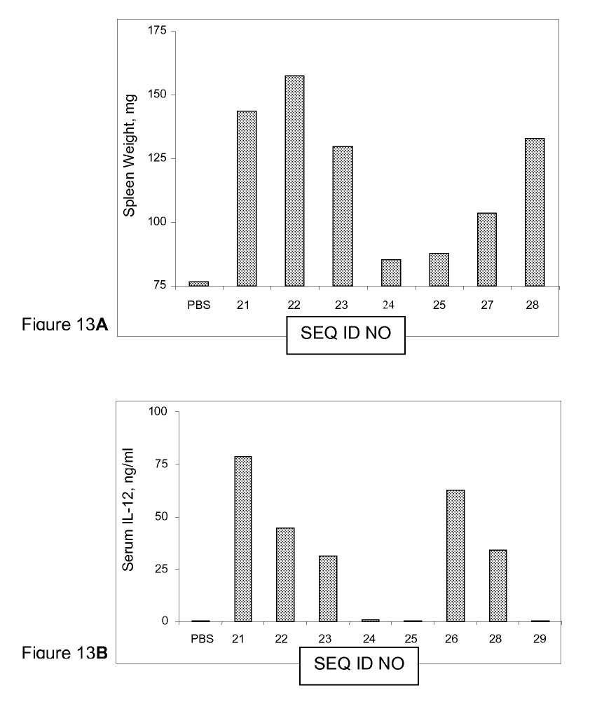

Splenomegaly (FIG. 13A) in C57BL/6 mice that received a 5 mg/kg dose of immune modulatory oligonucleotide, control compound, or PBS administered s.c. Change in spleen weights were determined 72 h after immune modulatory oligonucleotide administration. IL-12 (13.B) secretion in C57BL/6 mice induced by immune modulatory oligonucleotides following s.c. administration at a dose of 1 mg/kg. Blood was collected at 4 h after immune modulatory oligonucleotide administration and IL-12 levels in the serum were determined by ELISA. The increase in spleen weight of mice following CpG oligo administration is a measure of immune modulatory activity. Both mouse and human-specific immune modulatory oligonucleotides containing dF or 1-iso-dC showed spleen enlargement compared with mice that received control immune modulatory oligonucleotides 4 (SEQ ID NO 4) or 5 (SEQ ID NO 5) (FIG. 13A). Mice that received mouse-specific immune modulatory oligonucleotides 22 (SEQ ID NO 22) or 23 (SEQ ID NO 23), which have the dF or ψ-iso-dC modification, caused greater increases in spleen weight than did mice injected with human-specific immune modulatory oligonucleotides 27 (SEQ ID NO 27) or 28 (SEQ ID NO 28). These results also indicate that mice that received immune modulatory oligonucleotides 22 (SEQ ID NO 22) or 28 (SEQ ID NO 28), which have the dF modification, caused greater increases in spleen weight than did mice injected with immune modulatory oligonucleotides 23 (SEQ ID NO 23) or 27 (SEQ ID NO 27), which have the ψ-iso-dC modification, respectively. Further examination of in vivo cytokine induction profiles revealed that both mouse and human-specific immune modulatory oligonucleotides, which contained dF or ψ-iso-dC modifications, induced elevation of IL-12 in mice 4 h after immune modulatory oligonucleotide administration (FIG. 13B). As was seen in the splenomegaly assay, mouse-specific immune modulatory oligonucleotide 22 (SEQ ID NO 22) induced higher levels of IL-12 than did immune modulatory oligonucleotide 23 (SEQ ID NO 23). These results demonstrate that both the modifications (dF or i-iso-dC) are tolerated and activate TLR9 but the levels of immune response are different and that administration in vivo of immune modulatory oligonucleotides containing novel bases generates unique immune response profiles.

### Example 20

### Human B-Cell Proliferation Induced by Immune Modulatory Oligonucleotides

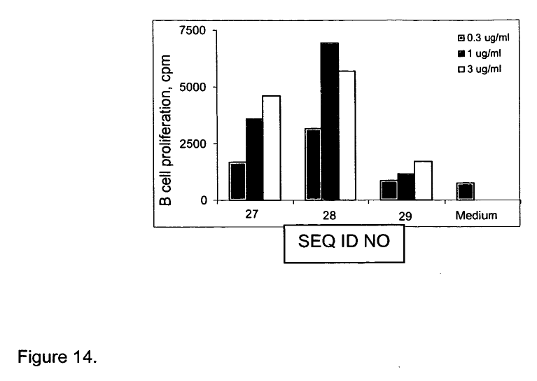

Human B-cells isolated from PBMC obtained from healthy human volunteers were stimulated with immune modulatory oligonucleotides at various concentrations and 3H-thymidine uptake was determined by scintillation counting (FIG. 14). FIG. 14 demonstrates that administration of immune modulatory oligonucleotides containing novel bases generates unique cell proliferation profiles, which vary with the base composition and the amount of the oligonucleotide administered.

### Example 21

### Cytokine/Chemokine Induction by Immune Modulatory Oligonucleotides

Induction of IL-2R, IL-6, IL-8, TNF-α, MIP-1α, MIP-β and MCP-1 were determined in human PBMC cell cultures by immune modulatory oligonucleotides 26 (SEQ ID NO 26), 27 (SEQ ID NO 27), 28 (SEQ ID NO 28), or control immune modulatory oligonucleotide 29 (SEQ ID NO 29) (Table 5).

TABLE 5

| SEQ | | | | | | | |
|---|---|---|---|---|---|---|---|
| ID | IL-2R | IL-6 | TNF-a | MIP-1a | MIP-b | MCP-1 | IL-8 |
| NO. | (pg/ml) | (pg/ml) | (pg/ml) | (pg/ml) | (pg/ml) | (pg/ml) | (pg/ml) |
| Me- | 96.05 | 13.66 | 14.48 | 34.18 | 191.42 | 18.51 | 125.28 |
| dium | | | | | | | |
| 26 | 178.21 | 523.42 | 165.00 | 115.41 | 1339.33 | 2036.87 | 1632.89 |
| 27 | 173.69 | 461.86 | 114.53 | 115.01 | 1225.94 | 406.18 | 3324.42 |
| 28 | 197.71 | 403.29 | 119.42 | 108.31 | 1121.74 | 443.07 | 3547.89 |
| 29 | 96.62 | 97.01 | 61.27 | 67.62 | 525.34 | 68.67 | 2769.96 |

### Example 22

### Immune Modulatory Oligonucleotides Containing dF or ψ-iso-dC in CpG Motif Activate Human PBMCs and B-Cells

The ability of immune modulatory oligonucleotides with dF or ψ-iso-dC modifications to activate human PBMCs and induce cytokine production was further examined. In these assays, immune modulatory oligonucleotides 27 (SEQ ID NO 27) and 28 (SEQ ID NO 28) were used, which contained a human-specific motif (Table 4A). Both immune modulatory oligonucleotide 27 (SEQ ID NO 27) and 28 (SEQ ID NO 28) induced IL-2R, IL-6, IL-8, TNF-α, MIP-1α, MIP-β and MCP-1 (Table 5) than did control 29 (SEQ ID NO 29), demonstrating that both modifications are tolerated and activate human TLR9. Both immune modulatory oligonucleotide 27 (SEQ ID NO 27) and 28 (SEQ ID NO 28) induced dose-dependent B-cell proliferation compared with control immune modulatory oligonucleotide 29 (SEQ ID NO 29) (FIG. 14).

### EQUIVALENTS

While the foregoing invention has been described in some detail for purposes of clarity and understanding, it will be appreciated by one skilled in the art from a reading of this disclosure that various changes in form and detail can be made without departing from the true scope of the invention and appended claims.

What is claimed is:

1. An immune modulatory oligonucleotide comprising at least one immune modulatory dinucleotide of the formula CG, wherein C is cytosine, 2′-deoxycytosine, N³-methyl-dC, dF or ψ-iso-dC, and G is guanosine, 2′-deoxyguanosine 2′-deoxy-7-deazaguanosine; arabinoguanosine or N¹-methyl-dG, provided that when C is cytosine or 2′-deoxycytosine, G is N¹-methyl-dG, and further provided that when G is guanosine or 2′-deoxyguanosine, C is N³-methyl-dC, dF or ψ-iso-dC.

2. The immune modulatory oligonucleotide according to claim 1 having a structure 5′-CTATCTGAC₁GTTCTCTGT-3′(SEQ ID NO: 1), 5′-CTATCTGACG₁TTCTCTGT-3′(SEQ ID NO: 2), 5′-CTATCTGTC₁GTTCTCTGT-3′(SEQ ID NO: 3), 5′-CTATCTGTCG₁TTCTCTGT-3′(SEQ ID NO: 4), 5′-TCTGAC₁GTTCT-X-TCTTGC₁AGTCT-5′(SEQ ID NO: 7), 5′-TCTGACG₁TTCT-X-TCTTG₁CAGTCT-5′(SEQ ID NO: 8), 5′-TCTGTC₁GTTCT-X-TCTTGC₁TGTCT-5′(SEQ ID NO: 9), 5′-TCTGTCG₁TTCT-X-TCTTG₁CTGTCT-5′(SEQ ID NO: 10); 5′-TCTGAC₂GTTCT-X-TCTTGC₂AGTCT-5′(SEQ ID NO: 22), 5′-TCTGAC₃GTTCT-X-TCTTGC₃AGTCT-5′(SEQ ID NO: 23), 5′-TCTGTC₃GTTCT-X-TCTTGC₃TGTCT-5′ (SEQ ID NO: 27), 5′-TC₃G₂AAC₃G₃TTC₃G₃-X-G₂C₃TTG₃C₃AAG₂C₃T-5′(SEQ ID NOS 30 and 34 respectively) or 5′-TCTGTC₂GTTCT-X-TCTTGC₂TGTCT-5′(SEQ ID NO: 28); wherein C₁=N³-methyl-dC; C₂=dF; C₃=ψ-iso-dC, G₁=N¹-methyl-dG; and X=glycerol linker.

3. A pharmaceutical formulation comprising the oligonucleotide according to claim 1 and a physiologically acceptable carrier.

4. A method for generating an immune response in a vertebrate, the method comprising administering to the vertebrate an immune modulatory oligonucleotide according to claim 1.

5. The method according to claim 4, wherein the route of administration is selected from parenteral, oral, sublingual, transdermal, topical, mucosal, inhalation, intranasal, aerosol, intraocular, intratracheal, intrarectal, vaginal, gene gun, dermal patch, eye drop and mouthwash.

6. The method according to claim 4, wherein the immune modulatory oligonucleotide is selected 5′-CTATCTGAC₁GTTCTCTGT-3′(SEQ ID NO: 1), 5′-CTATCTGACG₁TTCTCTGT-3′(SEQ ID NO: 2), 5′-CTATCTGTC₁GTTCTCTGT-3′(SEQ ID NO: 3), 5′-CTATCTGTCG₁TTCTCTGT-3′(SEQ ID NO: 4), 5′-TCTGAC₁GTTCT-X-TCTTGC₁AGTCT-5′(SEQ ID NO: 7), 5′-TCTGACG₁TTCT-X-TCTTG₁CAGTCT-5′(SEQ ID NO: 8), 5′-TCTGTC₁GTTCT-X-TCTTGC₁TGTCT-5′(SEQ ID NO: 9), 5′-TCTGTCG₁TTCT-X-TCTTG₁CTGTCT-5′(SEQ ID NO: 10); 5′-TCTGAC₂GTTCT-X-TCTTGC₂AGTCT-5′(SEQ ID NO: 22), 5′-TCTGAC₃GTTCT-X-TCTTGC₃AGTCT-5′(SEQ ID NO: 23), 5′-TCTGTC₃GTTCT-X-TCTTGC₃TGTCT-5′ (SEQ ID NO: 27), 5′-TC₃G₂AAC₃G₃TTC₃G₃-X-G₂C₃TTG₃C₃AAG₂C₃T-5′(SEQ ID NOS 30 and 34 respectively) or 5′-TCTGTC₂GTTCT-X-TCTTGC₂TGTCT-5′(SEQ ID NO: 28); wherein C₁═N³-methyl-dC; C₂=dF; C3=ψ-iso-dC, G₁=N¹-methyl-dG; and X=glycerol linker.

7. A method for therapeutically treating a vertebrate having cancer, an autoimmune disorder, airway inflammation, inflammatory disorders, skin disorders, allergy, asthma or a disease caused by a pathogen, such method comprising administering to the patient an immune stimulatory oligonucleotide according to claim 1.

8. The method according to claim 7, wherein the route of administration is selected from parenteral, oral, sublingual, transdermal, topical, intranasal, aerosol, intraocular, intratracheal, intrarectal, vaginal, gene gun, dermal patch, eye drop and mouthwash.

9. The method according to claim 7, wherein the immune modulatory oligonucleotide is selected from 5′-CTATCTGAC₁GTTCTCTGT-3′(SEQ ID NO: 1), 5′-CTATCTGACG₁TTCTCTGT-3′(SEQ ID NO: 2), 5′-CTATCTGTC₁GTTCTCTGT-3′(SEQ ID NO: 3), 5′-CTATCTGTCG₁TTCTCTGT-3′(SEQ ID NO: 4), 5′-TCTGAC₁GTTCT-X-TCTTGC₁AGTCT-5′(SEQ ID NO: 7), 5′-TCTGACG₁TTCT-X-TCTTG₁CAGTCT-5′(SEQ ID NO: 8), 5′-TCTGTC₁GTTCT-X-TCTTGC₁TGTCT-5′(SEQ ID NO: 9), 5′-TCTGTCG₁TTCT-X-TCTTG₁CTGTCT-5′(SEQ ID NO: 10); 5′-TCTGAC₂GTTCT-X-TCTTGC₂AGTCT-5′(SEQ ID NO: 22), 5′-TCTGAC₃GTTCT-X-TCTTGC₃AGTCT-5′(SEQ ID NO: 23), 5′-TCTGTC₃GTTCT-X-TCTTGC₃TGTCT-5′ (SEQ ID NO: 27), 5′-TC₃G₂AAC₃G₃TTC₃G₃-X-G₂C₃TTG₃C₃AAG₂C₃T-5′(SEQ ID NOS 30 and 34 respectively) or 5′-TCTGTC₂GTTCT-X-TCTTGC₂TGTCT-5′(SEQ ID NO: 28); wherein C₁═N³-methyl-dC; C₂=dF; C₃=ψ-iso-dC, G₁=N¹-methyl-dG; and X=glycerol linker.

10. A method for preventing cancer, an autoimmune disorder, airway inflammation, inflammatory disorders, skin disorders, allergy, asthma or a disease caused by a pathogen in a vertebrate, such method comprising administering to the vertebrate an immune stimulatory oligonucleotide according to claim 1.

11. The method according to claim 10, wherein the route of administration is selected from parenteral, oral, sublingual, transdermal, topical, mucosal, inhalation, intranasal, aerosol, intraocular, intratracheal, intrarectal, vaginal, gene gun, dermal patch, eye drop and mouthwash.

12. The method according to claim 10, wherein the immune modulatory oligonucleotide is selected from 5′-CTATCTGAC₁GTTCTCTGT-3′(SEQ ID NO: 1), 5′-CTATCTGACG₁TTCTCTGT-3′(SEQ ID NO: 2), 5′-CTATCTGTC₁GTTCTCTGT-3′(SEQ ID NO: 3), 5′-CTATCTGTCG₁TTCTCTGT-3′(SEQ ID NO: 4), 5′-TCTGAC₁GTTCT-X-TCTTGC₁AGTCT-5′(SEQ ID NO: 7), 5′-TCTGACG₁TTCT-X-TCTTG₁CAGTCT-5′(SEQ ID NO: 8), 5′-TCTGTC₁GTTCT-X-TCTTGC₁TGTCT-5′(SEQ ID NO: 9), 5′-TCTGTCG₁TTCT-X-TCTTG₁CTGTCT-5′(SEQ ID NO: 10); 5′-TCTGAC₂GTTCT-X-TCTTGC₂AGTCT-5′(SEQ ID NO: 22), 5′-TCTGAC₃GTTCT-X-TCTTGC₃AGTCT-5′(SEQ ID NO: 23), 5′-TCTGTC₃GTTCT-X-TCTTGC₃TGTCT-5′ (SEQ ID NO: 27), 5′-TC₃G₂AAC₃G₃TTC₃G₃-X-G₂C₃TTG₃C₃AAG₂C₃T-5′(SEQ ID NOS 30 and 34 respectively) or 5′-TCTGTC₂GTTCT-X-TCTTGC₂TGTCT-5′(SEQ ID NO: 28); wherein C₁═N³-methyl-dC; C₂=dF; C₃=ψ-iso-dC, G₁=N¹-methyl-dG; and X=glycerol linker.

13. The oligonucleotide according to claim 1, further comprising an antibody, antisense oligonucleotide, protein, antigen, allergen, chemotherapeutic agent or adjuvant.

14. The pharmaceutical composition according to claim 3, further comprising an antibody, antisense oligonucleotide, protein, antigen, allergen, chemotherapeutic agent or adjuvant.

15. The method according to claim 4, further comprising administering an antibody, antisense oligonucleotide, protein, antigen, allergen, chemotherapeutic agent or adjuvant.

16. The method according to claim 7, further comprising administering an antibody, antisense oligonucleotide, protein, antigen, allergen, chemotherapeutic agent or adjuvant.

17. The method according to claim 10, further comprising administering an antibody, antisense oligonucleotide, protein, antigen, allergen, chemotherapeutic agent or adjuvant.

18. An immune modulatory oligonucleotide compound, comprising an immune stimulatory dinucleotide of formula 5′-pyrimidine-purine-3′, wherein pyrimidine is N³-methyl-dC and purine is a natural or modified purine nucleoside.

19. An immune modulatory oligonucleotide compound, comprising an immune stimulatory dinucleotide of formula 5′-pyrimidine-purine-3′, wherein pyrimidine is a natural or modified pyrimidine nucleoside and purine is N¹-methyl-dG.

20. A pharmaceutical formulation comprising the oligonucleotide according to claim 18 and a physiologically acceptable carrier.

21. A pharmaceutical formulation comprising the oligonucleotide according to claim 19 and a physiologically acceptable carrier.

22. A method for generating an immune response in a vertebrate, the method comprising administering to the vertebrate an immune stimulatory oligonucleotide according to claim 18.

23. A method for generating an immune response in a vertebrate, the method comprising administering to the vertebrate an immune stimulatory oligonucleotide according to claim 19.

24. A method for therapeutically treating a vertebrate having cancer, an autoimmune disorder, airway inflammation, inflammatory disorders, skin disorders, allergy, asthma or a disease caused by a pathogen, such method comprising administering to the patient an immune stimulatory oligonucleotide according to claim 18.

25. A method for therapeutically treating a vertebrate having cancer, an autoimmune disorder, airway inflammation, inflammatory disorders, skin disorders, allergy, asthma or a disease caused by a pathogen, such method comprising administering to the patient an immune stimulatory oligonucleotide according to claim 19.

26. A method for preventing cancer, an autoimmune disorder, airway inflammation, inflammatory disorders, skin disorders, allergy, asthma or a disease caused by a pathogen in a vertebrate, such method comprising administering to the vertebrate an immune stimulatory oligonucleotide according to claim 18.

27. A method for preventing cancer, an autoimmune disorder, airway inflammation, inflammatory disorders, skin disorders, allergy, asthma or a disease caused by a pathogen in a vertebrate, such method comprising administering to the vertebrate an immune stimulatory oligonucleotide according to claim 19.

28. The oligonucleotide according to claim 18, further comprising an antibody, antisense oligonucleotide, protein, antigen, allergen, chemotherapeutic agent or adjuvant.

29. The pharmaceutical composition according to claim 20, further comprising an antibody, antisense oligonucleotide, protein, antigen, allergen, chemotherapeutic agent or adjuvant.

30. The method according to claim 22, further comprising administering an antibody, antisense oligonucleotide, protein, antigen, allergen, chemotherapeutic agent or adjuvant.

31. The method according to claim 24, further comprising administering an antibody, antisense oligonucleotide, protein, antigen, allergen, chemotherapeutic agent or adjuvant.

32. The method according to claim 26, further comprising administering an antibody, antisense oligonucleotide, protein, antigen, allergen, chemotherapeutic agent or adjuvant.

33. The oligonucleotide according to claim 19, further comprising an antibody, antisense oligonucleotide, protein, antigen, allergen, chemotherapeutic agent or adjuvant.

34. The pharmaceutical composition according to claim 21, further comprising an antibody, antisense oligonucleotide, protein, antigen, allergen, chemotherapeutic agent or adjuvant.

35. The method according to claim 23, further comprising administering an antibody, antisense oligonucleotide, protein, antigen, allergen, chemotherapeutic agent or adjuvant.

36. The method according to claim 25, further comprising administering an antibody, antisense oligonucleotide, protein, antigen, allergen, chemotherapeutic agent or adjuvant.

37. The method according to claim 27, further comprising administering an antibody, antisense oligonucleotide, protein, antigen, allergen, chemotherapeutic agent or adjuvant.

38. An immune stimulatory oligonucleotide compound, comprising an immune stimulatory dinucleotide of formula 5′-pyrimidine-purine-3′, wherein pyrimidine is N³-methyl-dC and purine is N¹-methyl-dG.

39. A pharmaceutical formulation comprising the oligonucleotide according to claim 38 and a physiologically acceptable carrier.

40. A method for generating an immune response in a vertebrate, the method comprising administering to the vertebrate an immune stimulatory oligonucleotide according to claim 38.

41. A method for therapeutically treating a vertebrate having cancer, an autoimmune disorder, airway inflammation, inflammatory disorders, skin disorders, allergy, asthma or a disease caused by a pathogen, such method comprising administering to the patient an immune stimulatory oligonucleotide according to claim 38.

42. A method for preventing cancer, an autoimmune disorder, airway inflammation, inflammatory disorders, skin disorders, allergy, asthma or a disease caused by a pathogen in a vertebrate, such method comprising administering to the vertebrate an immune stimulatory oligonucleotide according to claim 38.

43. The oligonucleotide according to claim 38, further comprising an antibody, antisense oligonucleotide, protein, antigen, allergen, chemotherapeutic agent or adjuvant.

44. The pharmaceutical composition according to claim 39, further comprising an antibody, antisense oligonucleotide, protein, antigen, allergen, chemotherapeutic agent or adjuvant.

45. The method according to claim 40, further comprising administering an antibody, antisense oligonucleotide, protein, antigen, allergen, chemotherapeutic agent or adjuvant.

46. The method according to claim 41, further comprising administering an antibody, antisense oligonucleotide, protein, antigen, allergen, chemotherapeutic agent or adjuvant.

47. The method according to claim 42, further comprising administering an antibody, antisense oligonucleotide, protein, antigen, allergen, chemotherapeutic agent or adjuvant.


SEQUENCE LISTING (36 sequences)

SEQ ID NO: 1 - DNA, 18 bp, Artificial Sequence
  Description of Artificial Sequence Synthetic oligonucleotide
ctatctgacgttctctgt

SEQ ID NO: 2 - DNA, 18 bp, Artificial Sequence
  Description of Artificial Sequence Synthetic oligonucleotide
ctatctgacgttctctgt

SEQ ID NO: 3 - DNA, 18 bp, Artificial Sequence
  Description of Artificial Sequence Synthetic oligonucleotide
ctatctgtcgttctctgt

SEQ ID NO: 4 - DNA, 18 bp, Artificial Sequence
  Description of Artificial Sequence Synthetic oligonucleotide
ctatctgtcgttctctgt

SEQ ID NO: 5 - DNA, 18 bp, Artificial Sequence
  Description of Artificial Sequence Synthetic oligonucleotide
ctatctgagcttctctgt

SEQ ID NO: 6 - DNA, 18 bp, Artificial Sequence
  Description of Artificial Sequence Synthetic oligonucleotide
ctatctgagcttctctgt

SEQ ID NO: 7 - DNA, 11 bp, Artificial Sequence
  Description of Artificial Sequence Synthetic oligonucleotide
tctgacgttct

SEQ ID NO: 8 - DNA, 11 bp, Artificial Sequence
  Description of Artificial Sequence Synthetic oligonucleotide
tctgacgttct

SEQ ID NO: 9 - DNA, 11 bp, Artificial Sequence
  Description of Artificial Sequence Synthetic oligonucleotide
tctgtcgttct

SEQ ID NO: 10 - DNA, 11 bp, Artificial Sequence
  Description of Artificial Sequence Synthetic oligonucleotide
tctgtcgttct

SEQ ID NO: 11 - DNA, 11 bp, Artificial Sequence
  Description of Artificial Sequence Synthetic oligonucleotide
tctgagcttct

SEQ ID NO: 12 - DNA, 11 bp, Artificial Sequence
  Description of Artificial Sequence Synthetic oligonucleotide
tctgagcttct

SEQ ID NO: 13 - DNA, 18 bp, Artificial Sequence
  Description of Artificial Sequence Synthetic oligonucleotide
ctatctgacgttctctgt

SEQ ID NO: 14 - DNA, 18 bp, Artificial Sequence
  Description of Artificial Sequence Synthetic oligonucleotide
ctatctgtcgttctctgt

SEQ ID NO: 15 - DNA, 17 bp, Artificial Sequence
  Description of Artificial Sequence Synthetic oligonucleotide
ctatctcaccttctctg

SEQ ID NO: 16 - DNA, 11 bp, Artificial Sequence
  Description of Artificial Sequence Synthetic oligonucleotide
tctgacgttct

SEQ ID NO: 17 - DNA, 11 bp, Artificial Sequence
  Description of Artificial Sequence Synthetic oligonucleotide
tctgacgttct

SEQ ID NO: 18 - DNA, 11 bp, Artificial Sequence
  Description of Artificial Sequence Synthetic oligonucleotide
tctcaccttct

SEQ ID NO: 19 - DNA, 11 bp, Artificial Sequence
  Description of Artificial Sequence Synthetic oligonucleotide
acacaccaact

SEQ ID NO: 20 - DNA, 11 bp, Artificial Sequence
  Description of Artificial Sequence Synthetic oligonucleotide
tctgtcgttct

SEQ ID NO: 21 - DNA, 11 bp, Artificial Sequence
  Description of Artificial Sequence Synthetic oligonucleotide
tctgacgttct

SEQ ID NO: 22 - DNA, 11 bp, Artificial Sequence
  Description of Artificial Sequence Synthetic oligonucleotide
tctgacgttct

SEQ ID NO: 23 - DNA, 11 bp, Artificial Sequence
  Description of Artificial Sequence Synthetic oligonucleotide
tctgacgttct

SEQ ID NO: 24 - DNA, 11 bp, Artificial Sequence
  Description of Artificial Sequence Synthetic oligonucleotide
tctgagcttct

SEQ ID NO: 25 - DNA, 11 bp, Artificial Sequence
  Description of Artificial Sequence Synthetic oligonucleotide
tctgagcttct

SEQ ID NO: 26 - DNA, 11 bp, Artificial Sequence
  Description of Artificial Sequence Synthetic oligonucleotide
tctgtcgttct

SEQ ID NO: 27 - DNA, 11 bp, Artificial Sequence
  Description of Artificial Sequence Synthetic oligonucleotide
tctgtcgttct

SEQ ID NO: 28 - DNA, 11 bp, Artificial Sequence
  Description of Artificial Sequence Synthetic oligonucleotide
tctgtcgttct

SEQ ID NO: 29 - DNA, 11 bp, Artificial Sequence
  Description of Artificial Sequence Synthetic oligonucleotide
acacaccaact

SEQ ID NO: 30 - DNA, 11 bp, Artificial Sequence
  Description of Artificial Sequence Synthetic oligonucleotide
tcgaacgttcg

SEQ ID NO: 31 - DNA, 11 bp, Artificial Sequence
  Description of Artificial Sequence Synthetic oligonucleotide
tcgaacgttcg

SEQ ID NO: 32 - DNA, 11 bp, Artificial Sequence
  Description of Artificial Sequence Synthetic oligonucleotide
tcgaacgttcg

SEQ ID NO: 33 - DNA, 11 bp, Artificial Sequence
  Description of Artificial Sequence Synthetic oligonucleotide
tcgaacgttcg

SEQ ID NO: 34 - DNA, 11 bp, Artificial Sequence
  Description of Artificial Sequence Synthetic oligonucleotide
tcgaacgttcg

SEQ ID NO: 35 - DNA, 11 bp, Artificial Sequence
  Description of Artificial Sequence Synthetic oligonucleotide
tctgtcgttct

SEQ ID NO: 36 - DNA, 11 bp, Artificial Sequence
  Description of Artificial Sequence Synthetic oligonucleotide
tctgtcgttct